(19) United States
(12) Patent Application Publication
(10) Pub. No.: US 2021/0353252 A1
(43) Pub. Date: Nov. 18, 2021

(54) METHOD FOR MAPPING THE VAULT FOR AN IMPLANTED INTER OCULAR LENS

(71) Applicant: Arcscan, Inc., Golden, CO (US)

(72) Inventors: Daniel Z. Reinstein, London (GB); Linda Johnson, Boulder, CO (US); Johan Erik Giphart, Aurora, CO (US); Andrew K. Levien, Morrison, CO (US); Matthew G. Sassu, Highlands Ranch, CO (US)

(21) Appl. No.: 17/318,572

(22) Filed: May 12, 2021

Related U.S. Application Data
(60) Provisional application No. 63/023,661, filed on May 12, 2020.

(51) Int. Cl. A61B 8/10; A61B 8/08; A61B 8/00; G06T 7/11; G06T 7/00; G06T 7/60

(52) U.S. Cl. CPC A61B 8/10; A61B 8/0833; A61B 8/469; A61F 2/16; G06T 7/0012; G06T 7/60; G06T 7/11

(57) ABSTRACT

The present disclosure is directed to a system and method that detects and measures a vault of an anterior segment of an eye of a patient. The system and method locate an implanted contact lens (ICL) between a cornea and a natural lens, form a B-scan of the eye based on the received ultrasound pulses, removes background pixels, such as by binarizing and thresholding, from the B-Scan from a grayscale color palette to a black/white color palette, determines, from the resulting B-scan a fiduciary location in the anterior segment of the eye, and forms using the resulting B-scan and fiduciary location, a vault map mapping a distance between an anterior segment surface and a posterior surface of the ICL along a plurality of lines drawn perpendicular to a local surface of the anterior segment surface.

### CROSS-REFERENCE TO RELATED APPLICATION

This application claims the benefits, under 35 U.S.C.§ 119(e), of U.S. Provisional Application Ser. No. 63/023,661 entitled “A Method for Mapping the Vault for an Implanted Inter Ocular Lens” filed May 12, 2020 which is incorporated herein by reference.

### FIELD OF INVENTION

The present invention relates to a method of ICL vault detection and measurement. This is accomplished using a precision ultrasound scanning device, for imaging the anterior segment of the eye; then automatically locating an implanted ICL and determining the position and orientation of the ICL with respect to a known fiducial within the eye.

### BACKGROUND OF THE INVENTION

Ultrasonic imaging has found use in accurate and reproducible measurements of structures of the eye, such as, for example, the cornea and lens capsule. Such measurements provide an ophthalmic surgeon valuable information that can be used to guide various surgical procedures, such as LASIK and lens replacement, for correcting refractive errors. They also provide diagnostic information after surgery has been performed to assess the geometrical location of corneal features such as the LASIK scar and lens features such as lens connection, position and orientation. This allows the surgeon to assess post surgical changes in the cornea or lens and to take steps to correct any problems that develop.

Except for on-axis measurements, dimensions and locations of eye components behind the iris cannot be fully determined by optical means. Ultrasonic imaging in the frequency range of about 5 MHz to about 80 MHz can be applied to make accurate and precise measurements of structures of the eye, such as the cornea, lens capsule, ciliary muscle, zonules and the like.

An arc scanner is an ultrasound scanning device utilizing a transducer that both sends and receives pulses as it moves along an arcuate guide track. The arcuate guide track has a center of curvature whose position can be moved to scan different curved surfaces. Later versions of arc scanners have mechanisms that allow the radius of curvature of the scanner to be changed. In this type of scanner, a transducer is moved along an arcuate guide track whose center of curvature can be changed and set approximately at the center of curvature of the eye surface of interest. The transducer generates many acoustic pulses as it moves along the arcuate guide track. These pulses reflect off specular surfaces and other tissue interfaces within the eye. Each individual return pulse is detected and digitized to produce a series of A-scans. The A-scans can then be combined to form a cross-sectional image of the eye features of interest. The image combining A-scans is commonly called a B scan.

At a center frequency of about 38 MHz, a typical arc scanner has an axial resolution of about 20 microns and a lateral resolution of about 150 microns. The reproducibility of arc scanner images is typically about 2 microns.

Ultrasonic imaging requires a liquid medium to be interposed between the object being imaged and the transducer, which requires, in turn, that the eye, the transducer and the path between them be at all times immersed in a liquid medium. Concern for safety of the cornea introduces the practical requirement that the liquid medium be either pure water or normal saline water solution. There are reasons to prefer that the medium be pure water or physiologic saline (also known as normal saline) but the embodiments do not exclude other media suitable for conducting acoustic energy in the form of ultrasound. Most other media present an increased danger to the patient's eye, even with a barrier interposed between the eye and the ultrasonic transducer. Barriers can leak or be breached, allowing the liquids on either side to mix, thus bringing a potentially harmful material into contact with the eye.

An eyepiece serves to complete a continuous acoustic path for ultrasonic scanning, that path extending from the transducer to the surface of the patient's eye. The eyepiece also separates the water in which the patient's eye is immersed from the water in the chamber in which the ultrasound transducer and guide track assembly are contained. Finally, the eyepiece provides a steady rest for the patient and helps the patient to remain steady during a scan. To be practical, the eyepiece should be free from frequent leakage problems, should be comfortable to the patient and its manufacturing cost should be low since it should be replaced for every new patient.

Another ultrasound scanning method is known as Ultrasound Bio Microscopy as embodied in a hand-held device commonly known as a UBM. A UBM can capture anterior segment images using a transducer to emit short acoustic pulses ranging from about 20 to about 80 MHz. This type of ultrasound scanner is also called a sector scanner.

A UBM is a hand-held ultrasonic scanner whose beams sweep a sector like a radar. The swept area is pie-shaped with its central point typically located near the face of the ultrasound transducer. In this type of scanner, an ultrasonic transducer oscillates about a fixed position so as to produce many acoustic echoes which are captured as a series of A-scans. These A-scans can then be combined to form a B-scan of a localized region of interest within the eye.

The UBM method is capable of making qualitative ultrasound images of the anterior segment of the eye but cannot make accurate, precision, comprehensive, measurable images of the cornea, lens or other components of the eye required for glaucoma screening, keratoconus evaluation or lens sizing. This is because of two reasons. First, the UBM is a hand-held device and relies on the steadiness of the operator's hand to maintain a fixed position relative to the eye being scanned for several seconds. Second, the UBM is pressed firmly onto the patient's eye to make contact with the patient's cornea to obtain good acoustic coupling. This gives rise to some distortion of the cornea and the eyeball.

Between these two limitations, the resolution is limited, at best, to the range of 40 to 60 microns and the reproducibility, at best, can be no better than 20 microns.

Optical Coherence Tomography (OCT) is a light-based imaging technology that can image the cornea although not to the full lateral extent as can an ultrasound instrument. OCT cannot see behind the scleral wall or behind the iris and is therefore of limited use in glaucoma screening. OCT does well for imaging the central retina although only to the lateral extent allowed by a dilated pupil. OCT images of the retina can disclose the damage caused by glaucoma. The approach of a precision ultrasound scanning device is to detect the onset of glaucoma by imaging structural changes in the anterior segment before any retinal damage occurs so that the disease can be identified and successfully treated with drugs and/or stents.

Ultrasonic imaging has been used in corneal procedures such as LASIK to make accurate and precise images and maps of cornea thickness which include epithelial thickness, Bowman's layer thickness and images of LASIK flaps.

New procedures such as implantation of accommodative lenses may provide nearly perfect vision without spectacles or contact lenses. Implantation of accommodative lenses requires precision measurements of, for example, the position and width of the natural lens for successful lens powering and implantation. Ultrasonic imaging can be used to provide the required accurate images of the natural lens especially where the zonules attach the lens to the ciliary body which is well off-axis and behind the iris and therefore not accessible to optical imaging.

Recent advances in ultrasonic imaging have allowed images of substantially the entire lens capsule to be made. This has opened up the ability of diagnostic devices to assist in both research of lens implantation devices and strategies, and to planning, executing and follow-up diagnostics for corrective lens surgery including specialty procedures such as glaucoma and cataract treatments as well as implantation of clear intraocular lenses including accommodative lens.

A phakic intraocular lens (PIOL) is a special kind of intraocular lens that is implanted surgically into the eye to correct myopia. It is called “phakic” (meaning “having a lens”) because the eye's natural lens is left untouched. Intraocular lenses that are implanted into eyes after the eye's natural lens has been removed during cataract surgery are known as pseudophakic. Phakic intraocular lenses are considered for patients with high refractive errors when laser options, such as LASIK and PRK are not the best surgical options.

PIOLS made of collamer (a foldable gel-like substance) requires a very small incision due the flexibility of the material. In the cases where refractive outcomes are not the best, LASIK can be used for fine-tuning. If a patient eventually develops a visually significant cataract, the PIOLs can be removed (explanted) when the patient undergoes cataract surgery.

### Speed of Sound in Different Regions of an Eye

Both ultrasound sector and ultrasound arc scanning instruments record time-of-arrival of reflected ultrasound pulses. A speed of sound of the medium is then used to convert these time of arrival measurements to distance measurements. Traditionally, a single representative speed of sound value is used. Usually the speed of sound of water at 37 C (1,531 m/s) is used although speeds of sound from 1,531 m/s to 1,641 m/s may be used (1,641 m/s is the speed of sound in a natural human lens).

The speed of sound varies in the different anterior segment regions of the eye such as the cornea, aqueous, natural lens and vitreous fluid. The speed of sound in these different regions have been measured by various researchers and are reasonably known. Therefore if the interfaces of these regions can be identified, the appropriate speeds of sounds for these regions can be used to convert times of arrivals to distances with more accuracy.

### Unintended Eye Motion and Instrument Motion During Scanning

It is also important to compensate for unintended patient head or eye motion because a scan of the anterior segment scan or lens capsule scan is typically made by overlaying two or three separate scans (such as an arcuate scan followed by two linear scans, also described in U.S. Pat. No. 9,597,059 entitled “Tracking Unintended Eye Movements in an Ultrasonic Scan of the Eye”.

Unintended patient eye motion includes saccades which are quick, simultaneous rotations of both eyes in the same direction involving a succession of discontinuous individual rotations of the eye orbit in the eye socket.

The speed of transducer motion in an precision scanning device such as described, for example, in U.S. Pat. No. 8,317,709, is limited because its movement is in a bath of water and excessive speed of motion of the transducer and its carriage can result in vibration of the entire instrument. In practice, a set of ultrasound scans can be carried out in about 1 to about 3 minutes from the time the patient's eye is immersed in water to the time the water is drained from the eyepiece.

The actual scanning process itself can be carried out in several tens of seconds, after the operator or automated software completes the process of centering and range finding. As is often the case, the patient may move his or her head slightly or may move his or her eye in its socket during this time. In some cases, the patient's heartbeat can be detected as a slight blurring of the images. If patient movements are large, the scan set can always be repeated.

### Creating Composite B-Scans

The arc scanning instrument of the present disclosure can create several distinct scan types. These are:
an arcuate scan having a fixed radius of curvature
a linear scan
a combined arcuate and linear scan allowing for various radii of curvature including inverse radii of curvature

These scans can be combined to form composite images because each image is formed from very accurate time-of-arrival data and transducer positional data. However, combining these separate scans into a composite scan must take into account patient eye movement during scanning; and instrument movement during scanning.

Due to the need for an eyes seal to provide a continuous medium for the ultrasound signal to travel between the transducer, any scanning device has a limitation in the range of movement the transducer can make relative to the eye. The range of the scanning device can be expanded to cover more of the anterior segment by introducing intentional and controlled eye movements and scanning the newly exposed portion of the eye that can now be reached. Registration techniques can be used to combine the scans of different eye positions to create a more complete composite image of the anterior segment of the eye.

U.S. patent application Ser. No. 16/422,182 entitled “Method for Measuring Behind the Iris after Locating the Scleral Spur” is pending. This application is directed towards a method for locating the scleral spur in an eye using a precision ultrasound scanning device for imaging of the anterior segment of the eye. One of the applications of a precision ultrasound scanning device or instrument is to image the region of the eye where the cornea, iris, sclera and ciliary muscle are all in close proximity. By using a knowledge of the structure of the eye in this region and employing binary filtering techniques, the position of the scleral spur can be determined. Once the position of the scleral spur is determined, a number of measurements that characterize the normal and abnormal shapes of components within this region of the anterior segment of the eye can be made.

There remains, therefore, a need for a method of ICL vault detection and measurement that may be accomplished by using a precision ultrasound scanning device for imaging the anterior segment of the eye. This method should include a means of automatically locating an implanted ICL and determining the position and orientation of the ICL with respect to a known fiducial within the eye.

### SUMMARY OF THE INVENTION

These and other needs are addressed by the present disclosure. The various embodiments and configurations of the present disclosure are directed generally to ultrasonic imaging of biological materials of an eye and in particular directed to a method of ICL vault detection and measurement, using a precision ultrasound scanning device, for imaging the anterior segment of the eye, for automatically locating an implanted ICL and determining the position and orientation of the ICL with respect to a known fiducial within the eye.

U.S. patent application Ser. No. 16/422,182 discloses a method, using ultrasonic imaging of the anterior segment of an eye, for automatically locating the scleral spur in the image using a form of segmentation analysis and, using the scleral spur as a fiduciary, automatically making measurements in front of and behind the iris from the modified image.

The method of U.S. patent application Ser. No. 16/422,182 is based on detecting a scleral spur in the ultrasound B-scan image of an eye of a patient. The method comprises providing an ultrasound device having a scan head with an arcuate guide track and a carriage movable along the arcuate guide track; an eyepiece configured to maintain the eye of the patient in a fixed location with respect to the arcuate guide track; and a transducer connected to the carriage. The method includes emitting, from the transducer, ultrasound pulses as the carriage moves along the arcuate guide track; storing the received ultrasound pulses on a non-transitory computer readable medium; forming, by at least one electronic device, a B-Scan of the eye of the patient based on the received ultrasound pulses; binarizing, by the at least one electronic device, the B-Scan from a grayscale color palette to a black/white color palette; determining, by the at least one electronic device, an average surface of a sclera of the eye; and locating, by the at least one electronic device, a bump of the average surface of the sclera that corresponds to the scleral spur.

In the present disclosure, the method of U.S. patent application Ser. No. 16/422,182 for detecting the scleral spur in a B-scan is extended to deliver measurements of an implanted ICL relative to the anterior surface of the natural lens.

The method of the present disclosure includes:
binarize the whole anterior segment image from 0 to 255 grades of grayscale to black and white.
determine background pixels and filter them out.
find the approximate center by starting in middle of image and trying to detect iris on each side. If found, choose mid-point between them, otherwise just use midpoint of image.
for each half, horizontally flip the right side image for processing.
find lens and ICL surface point at the mid column (right side of the half image).
trace along the surfaces in the left direction until one disappears.
create an object consisting of the cavity between the 2 surfaces, bounded on the right by the edge of the half image, and on the left by the end point of the first surface to “disappear.”
combine the 2 Regions of Interest (ROIs) into one.
for each column in the ROI, choose the bottom point of the ROI, find the point along a line perpendicular to the bottom point that intersects the top surface of the ROI. Record the bottom point and top point for each column, along with the distance between the two.

A map can be created by taking multiple B-scans of the eye (multiple meridians) and creating a vault map over an area of the anterior natural lens much like the thickness maps of the cornea that we have.

### Features Desired in an Image Used to Form a Vault Map

strong difference in pixel intensity between background pixels and real pixels.
continuous lines defining lens and ICL surface.
image mostly centered.
minimal extraneous miscellanea that can be confused with lens or ICL, or throw off calculations of background pixel intensity.

### Features Causing Difficulty in an Image Used to Form a Vault Map

broken surfaces on ICL or lens resulting in mistaking the top of the ICL is the bottom of the ICL.
missing ICL surface resulting in mistaking the top of the ICL is the bottom of the ICL.
extraneous features under the lens, resulting in mistaking the extraneous features for portions of the lens or ICL.

The result is a vault map showing numbers which are measurements in microns of the distance between the anterior lens and the posterior ICL along lines drawn perpendicular to the local surface of the anterior lens.

The preceding is a simplified summary of the invention to provide an understanding of some aspects of the invention. This summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below.

### The Following Definitions are Used Herein

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The phrases at least one, one or more, and and/or are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

An acoustically reflective surface or interface is a surface or interface that has sufficient acoustic impedance difference across the interface to cause a measurable reflected acoustic signal. A specular surface is typically a very strong acoustically reflective surface.

Anterior means situated at the front part of a structure; anterior is the opposite of posterior.

An A-scan is a representation of a rectified, filtered reflected acoustic signal as a function of time, received by an ultrasonic transducer from acoustic pulses originally emitted by the ultrasonic transducer from a known fixed position relative to an eye component.

An accommodative lens, also known as a presbyopic lens or presby lens, is an artificial intraocular lens that changes its focal distance in response to contraction of the ciliary body. When successfully implanted, an accommodative lens reverses presbyopia, the inability of the eye to change its focal distance from far to near.

Accuracy as used herein means substantially free from measurement error.

Aligning means positioning the acoustic transducer accurately and reproducibly in all three dimensions of space with respect to a feature of the eye component of interest (such as the center of the pupil, center of curvature or boundary of the cornea, lens, retina, etcetera).

The anterior chamber comprises the region of the eye from the cornea to the iris.

The anterior chamber depth also known as the “ACD” is minimum distance from the posterior cornea surface to the anterior lens surface.

The anterior segment comprises the region of the eye from the front of the cornea to the back of the lens.

Automatic refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Auto-centering means automatically, typically under computer control, causing centration of the arc scanning transducer with the eye component of interest.

In this disclosure, binarize means to convert grayscale pixels to black or white depending on which side of a selected grayscale threshold the pixel is on, where grayscale pixels range in values from 0 (black) to 255 (white).

Binary filtering or binary thresholding is used to transform an image into a binary image by changing the pixel values according to a selection rule. The user defines two thresholds and two intensity values. For each pixel in the input image, the value of the pixel is compared with the two thresholds. If the pixel value is inside the range defined by the two thresholds, the output pixel is assigned as an inside value. Otherwise the output pixels are assigned to an outside value.

A B-scan is a processed representation of A-scan data by either or both of converting it from a time to a distance using acoustic velocities and by using grayscales, which correspond to A-scan amplitudes, to highlight the features along the A-scan time history trace (the latter also referred to as an A-scan vector).

The ciliary body is the circumferential tissue inside the eye composed of the ciliary muscle and ciliary processes. There are three sets of ciliary muscles in the eye, the longitudinal, radial, and circular muscles. They are near the front of the eye, above and below the lens. They are attached to the lens by connective tissue called the zonule of Zinn, and are responsible for shaping the lens to focus light on the retina. When the ciliary muscle relaxes, it flattens the lens, generally improving the focus for farther objects. When it contracts, the lens becomes more convex, generally improving the focus for closer objects.

A composite image is an image that is made from the combination of multiple images merged onto a common co-ordinate system.

Compositing is the combining of images or image elements from separate sources into a single image. As used herein, compositing is achieved through digital image manipulation.

The term computer-readable medium as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the invention is considered to include a tangible storage medium or distribution medium and prior art-recognized equivalents and successor media, in which the software implementations of the present invention are stored.

Coronal means of or relating to the frontal plane that passes through the long axis of a body. With respect to the eye or the lens, this would be the equatorial plane of the lens which also approximately passes through the nasal canthus and temporal canthus of the eye.

The terms determine, calculate and compute, and variations thereof, as used herein, are used interchangeably and include any type of methodology, process, mathematical operation or technique.

Fiducial means a reference, marker or datum in the field of view of an imaging device.

Fixation means having the patient focus an eye on an optical target such that the eye's optical axis is in a known spatial relationship with the optical target. In fixation, the light source is axially aligned in the arc plane with the light source in the center of the arc so as to obtain maximum signal strength such that moving away from the center of the arc in either direction results in signal strength diminishing equally in either direction away from the center.

In this disclosure, grayscale means an image in which the value of each pixel is a single sample representing only intensity information. Images of this sort are composed exclusively of shades of gray, varying from black at the weakest intensity to white at the strongest intensity. Grayscale images are commonly stored with 8 bits per sampled pixel. This pixel depth allows 256 different intensities (shades of gray) to be recorded where grayscale pixels range in values from 0 (black) to 255 (white).

The home position of the imaging ultrasound transducer is its position during the registration process.

An ICL is an artificial lens that is implanted in the eye. It is an implantable contact lens inserted through a small incision in the eye and placed into its position behind the iris but in front of the natural lens.

Image stitching is the process of combining multiple B-scan images with overlapping fields of view to produce a composite B-scan.

An imaging ultrasound transducer is the device that is responsible for creating the outgoing ultrasound pulse and detecting the reflected ultrasound signal that is used for creating the A-Scans and B-Scans.

In this disclosure, isolating as applied to a binarized image means to isolate the scleral material containing the scleral spur from iris, cornea and ciliary material.

In this disclosure, a moving average (also referred to as a rolling average or running average) is a way of analyzing data points by creating a series of averages of different subsets of adjacent data points in the full data set.

The term module as used herein refers to any known or later developed hardware, software, firmware, artificial intelligence, fuzzy logic, or combination of hardware and software that is capable of performing the functionality associated with that element. Also, while the invention is described in terms of exemplary embodiments, it should be appreciated that individual aspects of the invention can be separately claimed.

Optical as used herein refers to processes that use light rays.

The optical axis of the eye is a straight line through the centers of curvature of the refracting surfaces of an eye (the anterior and posterior surfaces of the cornea and lens).

Phakic intraocular lenses, or phakic lenses, are lenses made of plastic or silicone that are implanted into the eye permanently to reduce a person's need for glasses or contact lenses. Phakic refers to the fact that the lens is implanted into the eye without removing the eye's natural lens. During phakic lens implantation surgery, a small incision is normally made in the front of the eye. The phakic lens is inserted through the incision and placed just in front of or just behind the iris.

Positioner means the mechanism that positions a scan head relative to a selected part of an eye. In the present disclosure, the positioner can move back and forth along the x, y or z axes and rotate in the β direction about the z-axis. Normally the positioner does not move during a scan, only the scan head moves. In certain operations, such as measuring the thickness of a region, the positioner may move during a scan.

Posterior means situated at the back part of a structure; posterior is the opposite of anterior.

The posterior chamber comprises the region of the eye from the back of the iris to the front of the lens.

The posterior segment comprises the region of the eye from the back of the lens to the rear of the eye comprising the retina and optical nerve.

Precise as used herein means sharply defined and repeatable.

Precision means how close in value successive measurements fall when attempting to repeat the same measurement between two detectable features in the image field. In a normal distribution precision is characterized by the standard deviation of the set of repeated measurements. Precision is very similar to the definition of repeatability.

The pulse transit time across a region of the eye is the time it takes a sound pulse to traverse the region.

Purkinje images are reflections of objects from structure of the eye. There are at least four Purkinje images that are visible on looking at an eye. The first Purkinje image (P1) is the reflection from the outer surface of the cornea. The second Purkinje image (P2) is the reflection from the inner surface of the cornea. The third Purkinje image (P3) is the reflection from the outer (anterior) surface of the lens. The fourth Purkinje image (P4) is the reflection from the inner (posterior) surface of the lens. Unlike the others, P4 is an inverted image. The first and fourth Purkinje images are used by some eye trackers, devices to measure the position of an eye. Purkinje images are named after Czech anatomist Jan Evangelista Purkynè (1787-1869).

Refractive means anything pertaining to the focusing of light rays by the various components of the eye, principally the cornea and lens.

Registration as used herein means aligning.

ROI means Region of Interest.

Saccades are quick, simultaneous rotations of both eyes in the same direction involving a succession of discontinuous individual rotations of the eye orbit in the eye socket. These rapid motions can be on the order of 20 degrees of rotation with a maximum velocity of 200 degrees/sec and are a part of normal eyesight.

Scan head means the mechanism that comprises the ultrasound transducer, the transducer holder and carriage as well as any guide tracks that allow the transducer to be moved relative to the positioner. Guide tracks may be linear, arcuate or any other appropriate geometry. The guide tracks may be rigid or flexible. Normally, only the scan head is moved during a scan.

The scleral spur in the human eye is an annular structure composed of collagen in the anterior chamber. The scleral spur is a fibrous ring that, on meridional section, appears as a wedge projecting from the inner aspect of the anterior sclera. The spur is attached anteriorly to the trabecular meshwork and posteriorly to the sclera and the longitudinal portion of the ciliary muscle.

A scout image is an image taken in order to find the anatomy of interest in preparation for a useable image showing the anatomy of interest. The scout image may be used or deleted as appropriate. A scout image or scout view is a preliminary image obtained prior to performing the major portion of a particular study and is used, for example, to plot the locations where the subsequent slice images will be obtained. Many radiologists consider CT scout images to be merely a guide for correlating the levels of axial images. Unfortunately, in many instances, those scout images show critical diagnostic information that is not displayed on the axial images, particularly in cranial, thoracic, and abdominal studies.

A scout film is a preliminary film taken of a body region before a definitive imaging study—e.g., a scout film of the chest before a CT. “Scouts” serve to establish a baseline and may be used before performing angiography, CT, or MRI.

Sector scanner is an ultrasonic scanner that sweeps a sector like a radar. The swept area is pie-shaped with its central point typically located near the face of the ultrasound transducer.

Segmentation analysis as used in this disclosure means manipulation of an ultrasound image to determine the boundary or location of an anatomical feature of the eye.

In this disclosure, smoothing as applied to a selected surface of a binarized image means to prepare the surface to be characterized by a straight line by removing protrusions above a first selected threshold and recesses below a second selected threshold. This is a special case of segmentation analysis.

The ciliary sulcus is the groove between the iris and ciliary body. The scleral sulcus is a slight groove at the junction of the sclera and cornea.

In the human eye, the scleral spur is an annular structure composed of collagen. It is a protrusion of the sclera into the anterior chamber. The scleral spur is the most anterior projection of the sclera internally. It is a circular ridge of sclera on the internal aspect of the corneoscleral junction. On cross-section, it appears as a hooklike process deep to the scleral venous sinus; relatively rigid, it provides attachment for the meridional fibers of the ciliary body.

The suprachoroid lies between the choroid and the sclera and is composed of closely packed layers of long pigmented processes derived from each tissue.

The suprachoroidal space is a potential space providing a pathway for uveoscleral outflow and becomes an actual space in choroidal detachment. The hydrostatic pressure in the suprachoroidal space is an important parameter for understanding intraocular fluid dynamics and the mechanism of choroidal detachment.

In this disclosure, thresholding means to select a threshold and divide objects into those above the threshold and those below the threshold.

A track or guide track is an apparatus along which another apparatus moves. In an ultrasound scanner or combined ultrasound and optical scanner, a guide track is an apparatus along which one or more ultrasound transducers and/or optical probes moves during a scan.

Ultrasonic means sound that is above the human ear's upper frequency limit. When used for imaging an object like the eye, the sound passes through a liquid medium, and its frequency is many orders of magnitude greater than can be detected by the human ear. For high-resolution acoustic imaging in the eye, the frequency is typically in the approximate range of about 5 to about 80 MHz.

An ultrasonic scanner is an ultrasound scanning device utilizing a transducer that both sends and receives pulses as it moves along 1) an arcuate guide track, which guide track has a center of curvature whose position can be moved to scan different curved surfaces; 2) a linear guide track; and 3) a combination of linear and arcuate guide tracks which can create a range of centers of curvature whose position can be moved to scan different curved surfaces.

As used herein, the vault is the three-dimensional space or volume between the anterior surface of the natural lens or capsule and the posterior surface of the ICL. A vector refers to a single acoustic pulse and its multiple reflections from various eye components. An A-scan is a representation of this data whose amplitude is typically rectified.

Zonules are tension-able ligaments extending from near the outer diameter of the crystalline lens. The zonules attach the lens to the ciliary body which allows the lens to accommodate in response to the action of the ciliary muscle.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

The preceding is a simplified summary of the disclosure to provide an understanding of some aspects of the disclosure. This summary is neither an extensive nor exhaustive overview of the disclosure and its various embodiments. It is intended neither to identify key or critical elements of the disclosure nor to delineate the scope of the disclosure but to present selected concepts of the disclosure in a simplified form as an introduction to the more detailed description presented below. As will be appreciated, other embodiments of the disclosure are possible utilizing, alone or in combination, one or more of the features set forth above or described in detail below.

### BRIEF DESCRIPTION OF THE DRAWINGS

The present disclosure may take form in various components and arrangements of components, and in various steps and arrangements of steps. The drawings are only for purposes of illustrating the preferred embodiments and are not to be construed as limiting the invention. In the drawings, like reference numerals may refer to like or analogous components throughout the several views.

FIG. 1 is a schematic of the principal elements of a prior art ultrasound eye scanning device such as described in U.S. Pat. No. 8,317,709.

FIG. 2 is a schematic cutaway drawing of an arc scanner showing a patient in place for scanning.

FIG. 3 is a schematic of a prior art eye seal first disclosed in U.S. Pat. No. 8,758,252.

FIG. 4 is a cutaway view of a prior art arc scanning device with patient in position for scanning.

FIG. 5 is an ultrasound image of an ICL with background pixels filtered out. This figure illustrates an ultrasound image of a patient's eye with an implanted ICL. The vault is the volume between the posterior surface of the ICL and the anterior surface of the natural lens.

FIG. 6 is an ultrasound image of the left half of the lens and ICL surfaces terminated at the midpoint. This figure is a thresholded ultrasound image of a patient's eye. The image is split at the visual axis and noise has been removed.

FIG. 7 is an ultrasound image showing the left-hand cavity between the 2 surfaces of FIG. 6. This figure depicts the region of interest which remains after analyzing the thresholded image (FIG. 6) and represents the initial estimate of the vault between the anterior surface of the lens capsule and the posterior surface of the ICL lens. This is the first half of the Vault.

FIG. 8 is an ultrasound image showing the right-hand cavity between the 2 surfaces of FIG. 6. This figure depicts the region of interest in the other half of the thresholded image (FIG. 6) representing the initial estimate of the right half of the vault. The same analysis is performed on right side as the left side.

FIG. 9 is an ultrasound image of the left and right hand surfaces combined. This figure represents the complete vault extracted from the thresholded image by combining the two areas found in the analysis.

FIG. 10 is an ultrasound image showing the bottom point and top point for each column, along with the distance between the two. This figure is a visual representation of the vault height measurements overlayed on an ultrasound image. Each line links a position on the anterior capsule with its corresponding point on the posterior ICL surface. The location of the point on the anterior capsule, combined with the distance between each point pair, (one on the capsule and its corresponding point on the ICL lens) forms the foundational information for a vault map. For clarity, only a subset of the calculated points is demonstrated here.

FIG. 11 is an ultrasound image illustrating features of images causing difficulty by showing broken ICL/lens that can be interpreted as the cornea mistaken for a lens.

FIG. 12 is an ultrasound image illustrating features of images causing difficulty by showing a top of an ICL was mistaken as the bottom of the ICL and shows possible errors in the vault map calculation. In this case, a section of the anterior ICL surface was mistaken for the posterior ICL surface.

FIG. 13 is another ultrasound image illustrating features of images causing difficulty by showing a top of an ICL that was mistaken as the bottom of the ICL.

FIG. 14 is another ultrasound image illustrating features of images causing difficulty by showing a top of an ICL that was mistaken as the bottom of the ICL. This figure shows another possible error in the vault map calculation. In this case, minor cataracts within the capsule were mistaken for the vault area of interest.

FIG. 15 is a vault map for an ICL implanted in an eye. The values shown in the figure are the vault width in microns at that location.

FIG. 16 is a post-op dry run. This figure is a post-op ICL report from the ArcScan Insight software demonstrating the standardized measurements taken in this zeroth meridian image. The rapid caliper tool guides the user through the measurement sequence, changes the caliper type as needed, and populates the table while the user only has to focus on clicking the correct landmarks.

FIG. 17 is page 1 of an ICL vault map report. This figure shows the vaults calculated from two different scans of the same eye on a given patient, as well as a third map showing the difference between them. If only one scan has been taken, only a single vault map will appear. Measurements from the rapid caliper tool are shown below the vault maps for each of the scan sets.

FIG. 18 is an ultrasound image of an ICL in an eye. The vault, which is the distance between the posterior surface of the ICL and the anterior surface of the natural lens, is shown as a short white vertical line.

FIG. 19 is a schematic representation of an ICL implanted in an eye. This figure illustrates an ICL positioned between the iris and the front of the natural lens. The vault is shown as a short black vertical line.

FIG. 20 is a flow chart of the principal steps used to generate a vault map from an ultrasound B-scan.

FIG. 21 is an example of a final vault map.

FIG. 22 shows other examples of final vault maps.

FIG. 23 is an ultrasonic image for detecting and analyzing various surfaces.

FIG. 24 is a block diagram of an embodiment of a computer or computing system environment operable to execute as the one or more devices described herein.

### DETAILED DESCRIPTION OF THE DRAWINGS

### Ultrasound Eye Scanning Apparatus

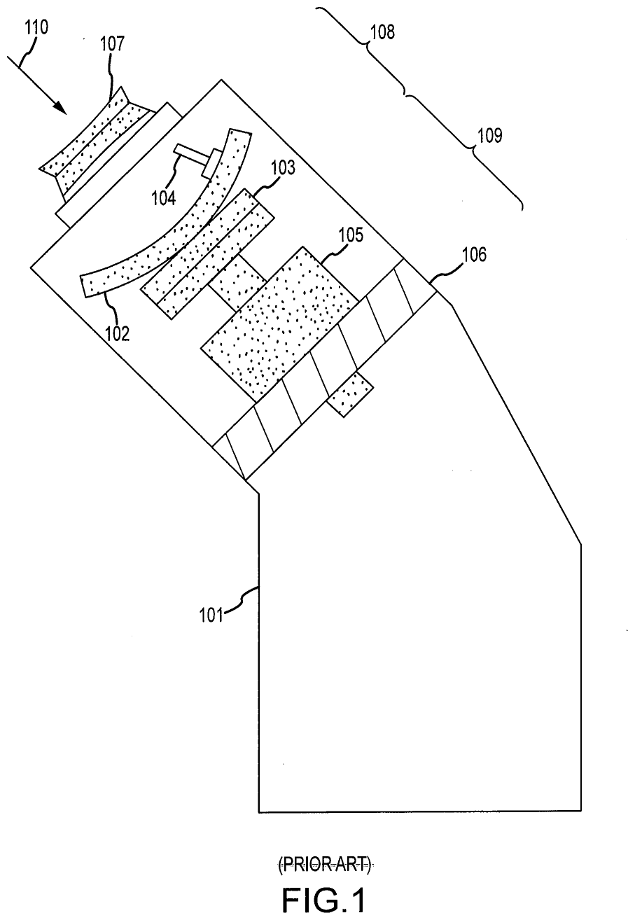

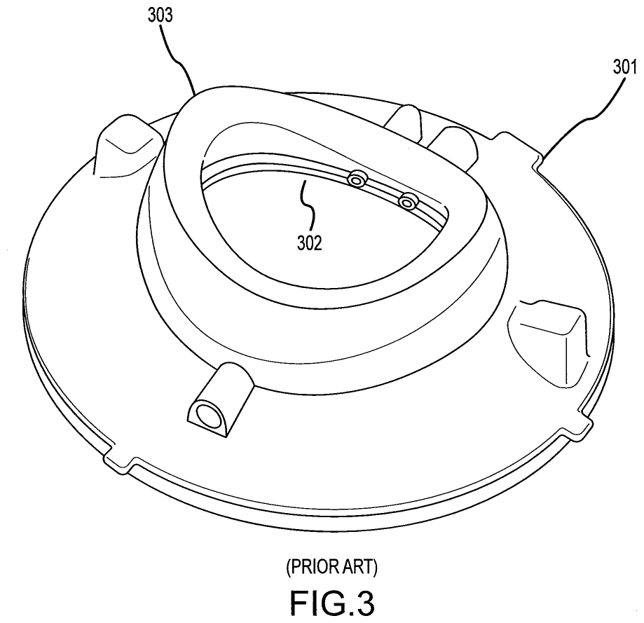

FIG. 1 is a schematic of the principal elements of a prior art ultrasound eye scanning device such as described in U.S. Pat. No. 8,317,709, entitled “Alignment and Imaging of an Eye with an Ultrasonic Scanner” which is incorporated herein by reference. The scanning device 101 of this example is comprised of a disposable eyepiece 107, a scan head assembly 108 and a positioning mechanism 109. The scan head assembly 108 is comprised of an arcuate guide track 102 with a scanning transducer 104 on a transducer carriage which moves back and forth along the arcuate guide track 102, and a linear guide track 103 which moves the arcuate guide track 102 back and forth. The positioning mechanism assembly 109 is comprised of an x-y-z and beta mechanisms 105 mounted on a base 106. The base 106 is rigidly attached to the scanning device 101. A longitudinal axis 110 passes generally through a center of the head assembly 108 and is substantially perpendicular to a face of the eyepiece 107. A video camera (not shown) may be positioned within the scanning device 101 and aligned with the longitudinal axis 110 to provide an image of a patient's eye through the eyepiece 107. The scanning device 101 is typically connected to a computer (not shown) which includes a processor module, a memory module, a keyboard, a mouse or other pointing device, a printer, and a video monitor. One or more fixation lights (not shown) may be positioned within the scanning device at one or more locations. The eyepiece 107 may be disposable as described in FIG. 3.

The positioning mechanism assembly 109 and scan head assembly 108 are both fully immersed in water (typically distilled water) which fills the chamber from base plate 106 to the top of the chamber on which the eyepiece 107 is attached.

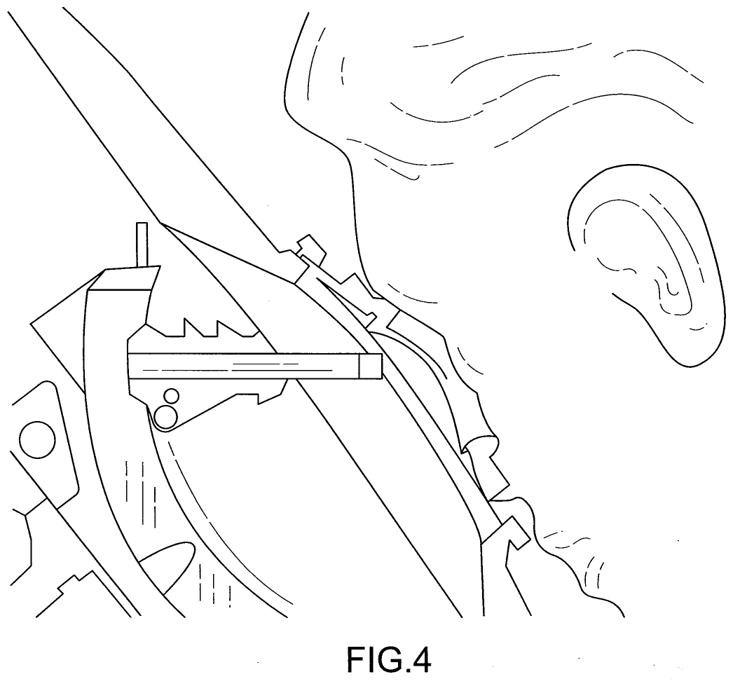

A patient is seated at the scanning device 101 with one eye engaged with the disposable eyepiece 107. The patient is typically directed to look downward at one of the fixation lights during a scan sequence. The patient is fixed with respect to the scanning device 101 by a headrest system such as shown, for example, in FIG. 4, and by the eyepiece 107.

An operator using a mouse and/or a keyboard and the video monitor, for example, inputs information into the computer selecting the type of scan and scan sequences as well as the desired type of output analyses. The operator using the mouse and/or the keyboard, the video camera located in the scanning machine, and the video screen, centers a reference marker such as, for example, a set of cross hairs displayed on the video screen on the desired component of the patient's eye which is also displayed on video screen. This is done by setting one of the cross hairs as the prime meridian for scanning. These steps are carried out using the positioning mechanism which can move the scan head in the x, x, z and beta space (three translational motions plus rotation about the z-axis). The z-axis is parallel to the longitudinal axis 110. Once this is accomplished, the operator instructs the computer to proceed with the scanning sequence. Now the computer processor takes over the procedure and issues instructions to the scan head 108 and the scanning transducer 104 and receives positional and imaging data. The computer processor proceeds with a sequence of operations such as, for example: (1) with the transducer carriage substantially centered on the arcuate guide track, rough focusing of the scanning transducer 104 on a selected eye component; (2) accurately centering of the arcuate guide track with respect to the selected eye component; (3) accurately focusing the scanning transducer 104 on the selected feature of the selected eye component; (4) rotating the scan head assembly 108 through a substantial angle (including orthogonal) and repeating steps (1) through (3) on a second meridian; (5) rotating the scan head back to the prime meridian; (6) initiating a set of A-scans along each of the of selected scan meridians, storing this information in the memory module; (7) utilizing the processor, converting the A-scans for each meridian into a set of B-scans and then processing the B-scans to form an image associated with each meridian; (8) performing the selected analyses on the A-scans, B-scans and images associated with each or all of the meridians scanned; and (9) outputting the data in a preselected format to an output device such as a printer. As can be appreciated, the patient's head must remain fixed with respect to the scanning device 101 during the above operations when scanning is being carried out, which in a modern ultrasound scanning machine, can take several tens of seconds.

An eyepiece serves to complete a continuous acoustic path for ultrasonic scanning, that path extending in water from the transducer to the surface of the patient's eye. The eyepiece 107 also separates the water in which the patient's eye is immersed (typically a saline solution) from the water in the chamber (typically distilled water) in which the transducer guide track assemblies are contained. The patient sits at the machine and looks down through the eyepiece 107 in the direction of the longitudinal axis 110. Finally, the eyepiece provides an additional steady rest for the patient and helps the patient's head to remain steady during a scan procedure.

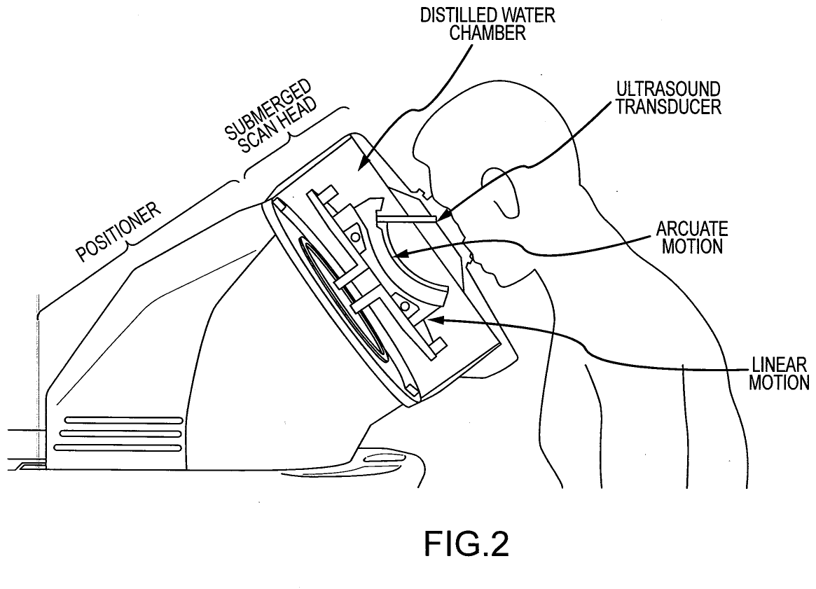

FIG. 2 is a schematic cutaway drawing of an arc scanner. The scan head is immersed in distilled water within a bucket (shown in cut away view). The bucket is attached to and separated from a housing which contains the instrument electronics and the positioner mechanism. The housing is open to ambient air. A telescoping shaft of the positioner mechanism goes through a large, flexible sealing membrane into the bucket and the scan head is attached to the immersed end of this shaft. The scan head comprises a linear track on which is mounted an arcuate track. An ultrasound transducer is mounted on a carriage which can move along the arcuate track. In FIG. 2, a patient is shown with one eye pushed against an eye piece such as shown in more detail below in FIG. 3.

FIG. 3 is a schematic of a prior art eye seal first disclosed in U.S. Pat. No. 8,758,252 entitled “Innovative Components for an Ultrasonic Arc Scanning Apparatus”.

An eyepiece serves to complete a continuous (substantially constant acoustic impedance) acoustic path for ultrasonic scanning, that path extending from the transducer to the surface of the patient's eye. The eyepiece also separates the water in which the patient's eye is immersed from the water in the chamber in which the positioner and scan head assemblies are immersed. Finally, when the patient is in position for a scan with his or her head firmly against the eye piece, the eyepiece provides a reference frame for the patient and helps the patient's head to remain steady during a scan. The eyepiece also must be able to pass optical wavelengths of light so that fixation targets can be used to focus the patient's eye in a desired focal state and alignment with respect to the eye's visual or optical axis.

An eyepiece system that satisfies these requirements typically consists of a mounting ring and a detachable eye piece. The mounting ring is attached to and is typically a permanent part of the main arc scanner assembly. The mounting ring has several attachment grooves which can accept attaching mechanisms on the eye piece. The eye piece is comprised of a base and a soft rubber or foam contoured face seal which is designed to seal against a typical human face around the eye that is to be scanned.

The eyepiece consists of a base ring 301 and a soft flexible sealing ring 303. A mounting ring (not shown) is attached to the main scanner housing as a permanent part of the main scanner assembly. The mounting ring has several attachment grooves which can accept attaching tabs molded into the eye piece base ring 301. In this embodiment, the attaching tabs are pushed down into the attachment grooves and then rotated into position, using the thumb and finger protrusions also molded into the eye piece base ring 301 to form a mechanical connection that seals the eye piece base against the mounting ring to prevent water leakage. This is also known as a bayonet type connection.

A sealed hygienic barrier or membrane 302 is formed as part of the eye piece base 301 and is typically located where the soft rubber or foam face seal 303 is connected to the eye piece base 301.

The eye piece of FIG. 3 is further described in U.S. Pat. No. 8,758,252 entitled “Innovative Components for an Ultrasonic Arc Scanning Apparatus” and published application US2015/00238166 entitled “Disposable Eyepiece System for an Ultrasonic Eye Scanning Apparatus”, both of which are incorporated herein by reference.

FIG. 4 is a cutaway view of a prior art arc scanning device with patient in position for scanning. The patient's eye socket is pressed against the soft flexible sealing ring of the eye piece, which is now attached to the mounting ring which, in turn, is attached to the main scanner housing. An ultrasound probe is shown at the top end of the arcuate guide track and is aimed at the patient's eye. As the probe moves along the arcuate guide track, its long axis remains approximately perpendicular to the surfaces of the cornea and anterior lens of the patient's eye. The tip of the ultrasound transducer comes very close to the membrane that separates the distilled water in the instrument bucket from the saline solution in the eye piece. The transducer is attached to the transducer carriage by small magnets which will release the probe if it contacts the membrane with enough force to endanger the patient's eye.

### ICLs (Implantable Collamer Lens)

An ICL is an artificial lens that is implanted in the eye. It is an implantable contact lens inserted through a small incision in the eye and placed into its position behind the iris but in front of the natural lens.

These are also known as the Implantable Collamer® Lens. For example, the Visian ICL is FDA approved implantable lens that works with the natural eye to correct vision. The Visian ICL procedure does not remove corneal tissue. The lens gently unfolds when implanted in the eye, rests behind the iris, and is biocompatible with body chemistry.

Implantable contact lenses are thin, pliable lenses often used as an alternative to LASIK vision correction surgery for permanent vision improvement. The lenses are implanted in the eye during ICL eye surgery and work with the eye's natural lens to improve vision.

Unlike LASIK surgery, no laser is required for ICL eye surgery and the procedure can be reversed by performing an additional surgery to remove the lens.

Two types of lenses are available for ICL eye surgery. A foldable lens is inserted through a small incision and unfolds into its place between the iris and the eye's natural lens. This procedure requires an extremely small incision that is self-healing. Another available lens, on the other hand, is inserted in front of the iris through a somewhat larger incision that must be closed with sutures which dissolve over time.

Phakic intraocular lenses (IOLs), also known as implantable contact lenses (ICLs), are implantable contact lenses that are surgically inserted into the eye where they provide excellent quality of vision with predictable and stable results. The Visian ICL is a phakic intraocular lens receiving approval from the FDA for a wide range of myopic (nearsightedness) correction needs.

This technology is adapted from the proven lens technology used for cataract surgery, and works by placing the ICL in front of the natural lens inside the eye. The Visian ICL is made of a material that is biocompatible and provides excellent optical performance. The implantable contact lens (ICL) is a posterior chamber phakic IOL can be folded and injected through about a 3-mm self-sealing clear-corneal incision under topical anesthesia.

Perfectly fitting the ICL in the posterior chamber depends on lens design and lens sizing. These factors determine the position of the ICL in the posterior chamber, especially its vaulting—the phakic lens should neither touch the natural lens nor obstruct the normal circulation of the aqueous humor.

The concern about whether the ICL touches the crystalline lens has received much attention, because intimate contact between the artificial and the natural lenses raises the possibility of cataractogenesis.

Ultrasound biomicroscopy is helpful to identify the position of the posterior phakic IOL. Preoperatively, ultrasound can also show the existence of iridociliary cysts and other disorders that cannot be seen through slit lamp examination and would interfere with the ICL's normal position.

In a previous ultrasound study featuring a version of an ICL for the correction of high myopia, it was observed that the ICL clearly vaulted over the crystalline lens centrally; however, it touched the crystalline lens in mid-periphery in most cases, under the thickest part of the ICL, which is located at the optic-haptic junction (the vault is the distance between the posterior surface of the ICL and the anterior surface of the natural lens).

Contact between the ICL and the crystalline lens in midperiphery does not cause prompt opacification; however, it may block the normal circulation of the aqueous humor. A pool of aqueous stagnation may be responsible for the anterior subcapsular opacification seen in a few cases after uneventful implantation in the late postop period.

It is important to state that no ICL-induced cataracts have been reported with the hyperopic model, most likely because of its design, which avoids blockage of normal aqueous circulation.

Ideally, the ICL should bend forward, leaving a space between the phakic and crystalline lenses. However, ICL vaulting must not cause excessive contact with the posterior surface of the iris, which could lead to pigment dispersion, increasing the risk of developing glaucoma, especially in highly myopic eyes.

So proper ICL sizing is of paramount importance as it dictates vaulting and positioning of the ICL in the posterior chamber. ICL sizing calculation is crucial. Traditionally, sizing is based on the white-to-white measurement, which is an indirect and less accurate assessment of the ciliary sulcus diameter and can be misleading in some cases.

A more precise and direct way to measure the ciliary sulcus diameter to improve ICL sizing determination is needed Instead of using the recommended “golden rule,” which is to add 0.5 mm to the horizontal white-to-white distance for the myopic model and to subtract the same amount for the hyperopic one.

Small sizing miscalculations can bring about great errors in ICL positioning, and this “golden rule” has been criticized for lack of accuracy.

In adult life there is a steady axial growth of the crystalline lens of about 25 μm per year, which may be responsible for vaulting reduction over time, with all the negative consequences. That is why higher vaulting values are welcome, such as the recently recommended range of 300 μm to 600 μm.

Should cataract occur, the solution is simple because the ICL is easily explanted through the original clear-corneal incision. Routine phacoemulsification and IOL implantation are then performed, which, in reality, represent an alternative form of treatment, especially for high myopia in adults.

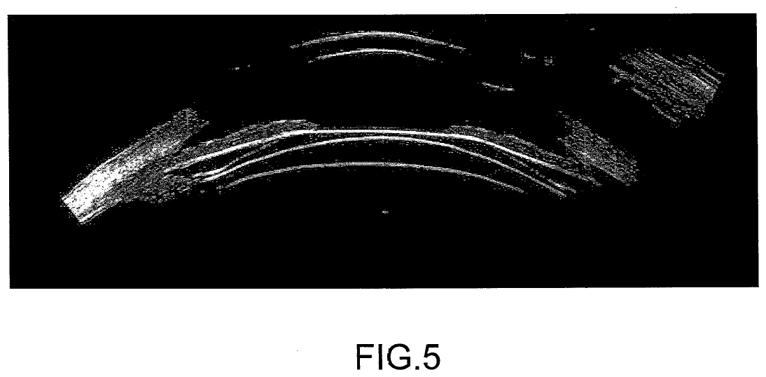

FIG. 5 is an ultrasound image of an ICL implanted in an eye. This figure shows the ICL positioned between the iris and the front of the natural lens. The vault, which is the distance between the posterior surface of the ICL and the anterior surface of the natural lens, is shown as a short white vertical line.

Two types of lenses are available for ICL eye surgery. A foldable lens is inserted through a small incision and unfolds into its place between the iris and the eye's natural lens. This procedure requires an extremely small incision that is self-healing. Another available lens, on the other hand, is inserted in front of the iris through a somewhat larger incision that must be closed with sutures which dissolve over time.

### The Vault

The vault is defined as the space between the posterior surface of the ICL and the anterior surface of the natural lens. Perfectly fitting the ICL in the posterior chamber depends on lens design and lens sizing. These factors determine the position of the ICL in the posterior chamber, especially its vaulting. The phakic lens should neither touch the natural lens nor obstruct the normal circulation of the aqueous humor.

Roadmap for Measuring and Detecting the ICL Vault

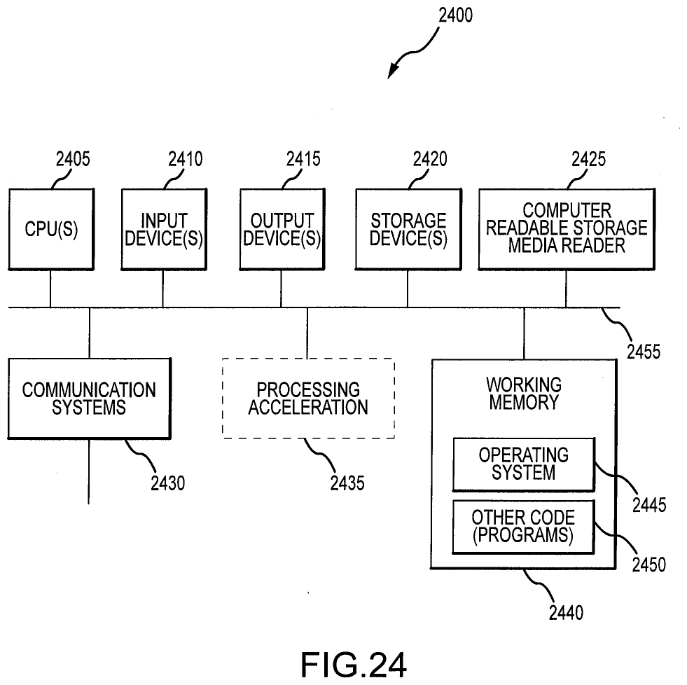

FIG. 24 illustrates one embodiment of a computer system 2400 that may be connected to the ultrasound eye scanning device and upon which servers or other systems described herein may be deployed or executed. The computer system 2400 is shown comprising hardware elements that may be electrically coupled via a bus 2455. The hardware elements may include one or more central processing units (CPUs) 2405; one or more input devices 2410 (e.g., a mouse, a keyboard, etc.); and one or more output devices 2415 (e.g., a display device, a printer, etc.). The computer system 2400 may also include one or more storage devices 2420. By way of example, storage device(s) 2420 may be disk drives, optical storage devices, solid-state storage device, such as a random access memory (“RAM”) and/or a read-only memory (“ROM”), which can be programmable, flash-updateable and/or the like.

The computer system 2400 may additionally include a computer-readable storage media reader 2425; a communications system 2430 (e.g., a modem, a network card (wireless or wired), an infra-red communication device, etc.); and working memory 2440, which may include RAM and ROM devices as described above. In some embodiments, the computer system 2400 may also include a processing acceleration unit 2435, which can include a DSP, a special-purpose processor and/or the like.

The computer-readable storage media reader 2425 can further be connected to a computer-readable storage medium, together (and, optionally, in combination with storage device(s) 2420) comprehensively representing remote, local, fixed, and/or removable storage devices plus storage media for temporarily and/or more permanently containing computer-readable information. The communications system 2430 may permit data to be exchanged with the network 2420 and/or any other computer described above with respect to the system 2400. Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information.

The computer system 2400 may also comprise software elements, shown as being currently located within a working memory 2440, including an operating system 2445 and/or other code 2450, such as program code implementing the servers or devices described herein. It should be appreciated that alternate embodiments of a computer system 2400 may have numerous variations from that described above. For example, customized hardware might also be used and/or particular elements might be implemented in hardware, software (including portable software, such as applets), or both. Further, connection to other computing devices such as network input/output devices may be employed.

The following description is based on processor executable MatLab code, which is stored in working memory 2440 and when executed by one or more central processing units (CPUs) or processors 2405 measures a vault for an ICL implanted in an eye of a patient and generates a vault map. While the present disclosure is discussed with reference to MatLab, it is to be understood that any programming code may be employed.

The processor executable function MeasureICLVault.m contains the main “entry point” from the Insight application of the present disclosure (also referenced herein as MeasureICLVault( ). This processor executable function determines a best guess at a background pixel intensity and column of the mid-point of the eye. The processor executable function calls or invokes the processor executable code DetermineICL_ROI_Object( ) to obtain the right and left regions of interest or ROI (ROI—the inverted vault space, spanning from the mid-column to the end of the lens and/or ICL surface), recombines the 2 ROIs, and then calls or invokes the processor executable code CalcTotalVaultUsingSlopeMethod( ) to measure the ICL vault from the combined ROI and GrayScale Image. The last function call in this processor executable function (DisplayVaultMeasurements( )) can be called for debugging purposes. The processor executable function returns the estimated midpoint as a 2-cell array representing the row and column of the estimated mid column along the top ICL surface. It also returns 4 arrays, all indexed by the pixel column (of the lens) in the original image. The arrays include the following:
vaultLensRow Array—represents the row in each column where the lens was detected. If no lens is detected in a row, a value is set to “0”.
vaultICLRow Array, vaultICLColArray—represents the row and column of the point found along the line perpendicular to the lens intersecting the back ICL surface. The index of these arrays represents the column of the associated point on the lens. If no point is found on the ICL surface, the values are set to 0.
vaultMeasurementArray—contains the measured vault for each point on the lens, in um.

The processor executable function FindMidColAndBackgroundPix.m calls DetectICLBackGroundPixel( ) to determine an intensity of background pixel and thresholds image accordingly. The processor executable function attempts to detect an ICL front surface and the iris on each side of eye. If two irises are detected, the processor executable function calculates the mid-column as halfway between the 2 irises. (This calculation does not need to be precise—it is only to try to definitively locate a point somewhere between the 2 irises so that when we “cut the image in half”, both sides will have a sufficient area of ICL surface without an iris.) If both irises are not detected, the processor executable function uses a mid-column of the entire image as the mid-column.

The processor executable function DetectICLBackgroundPixel.m determines the background intensity using 3 methods. This file/function is based on the processor executable code (DetectBackgroundPixel( ) with the following differences:
In using the jpg files output from the Insight Application to test with, it appeared that the first 20 rows and the first 25 columns were filled with some sort of miscellanea that needed to be ignored.
In method 2, the maximum intensity should be about 3 times the mean intensity versus
added processor executable code discounts a potential outlier of the output from the 3 methods.

The processor executable function MaskICLJunk.m, MaskLeftICLJunk.m attempts to mask out anything other than iris, sclera, ICL, and lens.

The processor executable function DetermineICL_ROI_Object.m isolates/returns an object representing the inverted vault space for the “half image” (space between the lens and the back of the ICL). It can accomplish this by:

1) Adjusting a threshold until separate lens/ICL surfaces are visible at the previously determined mid-column (which is the right edge of the objects in the half images)
2) Continue adjusting threshold until a vault object is isolated. The processor executable function calls the processor executable code FindICLLensSegment( ) to try to distinguish the ICL and lens surfaces from the cornea and cataract surfaces. If it cannot isolate the lens/ICL surfaces, it adjusts the threshold until it can (or gives up.) The processor executable function returns the ROI image, the gray scale image with all but the ROI area masked out, the final threshold used when obtaining ROI, and the left-most column of the ROI.

The processor executable function CalculateTotalVaultUsingSlopeMethod.m uses the ICL ROI as a starting point/guide, calls DeterminGrayPeaks( ) to find the surface peaks in the grayscale image for the back ICL and lens surfaces. The processor executable function then calls the processor executable code MeasureVaultUsingPeakSurfaces( ) to calculate the vault. Last, the processor executable function attempts to fill in holes in the lens and ICL arrays via interpolation, and then recalculates the vault measurement for any holes in that array.

The processor executable function DetermineGrayPeaks.m, for each column in a grayscale image, between specified start and end columns, calls the processor executable function FindLensAndICLSurfaces( ) to find the ICL and lens surfaces (peaks) using the ICL ROI as a guide. If a surface is not detected for 5 or more contiguous columns, the processor executable function resets the last known row for that surface to try to re-detect it, in case there was a gap in the surface (in the image . . . ) Once the processor executable function reaches the end of the ROI, it calls ExtendIGrayPeaks( ) to try to detect the surfaces for as far as it can. If there is not a valid ROI, the processor executable function starts at startCol and calls ExtendIGrayPeaks( ) to find as much of the surfaces as it can.

The processor executable function FindLensAndICLSurfaces( ) finds the row of the ICL surface and the row of the lens surface in a specified column of the image. If the bottom and top of the ICL ROI can be found, the processor executable function looks for the next peak above/below those rows in GrayCol (specified single column from the grayscale image).

The processor executable function ExtendIGrayPeaks( ) using the grayscale image, starts with last known row for the given surface, and traces the peak out further until it loses it.

The processor executable function FindPeaksInColumn.m finds the peaks (local maxima) in a specified column of a grayscale image between the specified starting and ending rows using the following logic:
If endingRow<=0, it signifies that we are only looking for first peak.
If consecutive cells are equivalent (and a peak), chooses the middle row.
If we find one peak, then dip below a “low threshold”, but don't dip below a valley threshold, and then find another equal peak within a specified range choose mid points between those 2 peaks.
   Returns an array of peak locations (peakRows), an associated array of the intensities at those peak locations, and the original column array with all but the peak locations set to zero.

The processor executable function MeasureVaultUsingPeakSurfaces.m, for each column along the lens surface, finds the average slope of the lens at that point (averages the slopes of lines formed by equidistant points on the lens from the current lens point, with various spans), finds the point on the ICL surface along the line perpendicular to the lens point, performs a reasonableness check on the point, records the point in the output arrays, and records the distance between the lens point and the associated ICL point. Outputs are the 4 arrays described in the processor executable functions CalculateTotalVaultUsingSlopeMethod( ) and MeasureICLVault( ). Finds a point the general area of the iris root. Not the exact location of the root, but somewhere close enough to establish a reasonable “region of interest” in the image.

### ICL Vault Detection/Measurement

U.S. patent application Ser. No. 16/422,182 entitled “A Method for Measuring Behind the Iris after Locating the Scleral Spur” is incorporated herein by reference.

U.S. patent application Ser. No. 16/422,182 discloses a method, using ultrasonic imaging of the anterior segment of an eye, for automatically locating the scleral spur in an ultrasound image of an eye using a form of segmentation analysis, and, using the scleral spur as a fiduciary, automatically making measurements from the image, in front of and behind the iris.

The method of U.S. patent application Ser. No. 16/422,182 consists of the following principal steps which are performed automatically:
1. Acquire B-Scans.
2. Binarize B-Scans.
3. Determine the iris/lens contact distance (ILCD) and anterior chamber depth (ACD).
4. Locate Root of the Iris.
5. Locate Root of the Ciliary Sulcus.
6. Isolate the Sclera.
7. Locate the Scleral Spur.
8. Using the scleral spur as a fiduciary, make measurements including, at least, the trabecular/iris angle (TIA), the iris lens contact distance, the iris zonule distance (IZD) and the trabecular ciliary process distance (TCPD).
9. Prepare an Automated based on a B-Scan with all measurements displayed.

The method of U.S. patent application Ser. No. 16/422,182 is based on detecting a scleral spur in an ultrasound image of an eye of a patient. The method comprises providing an ultrasound device having a scan head with an arcuate guide track and a carriage movable along the arcuate guide track; an eyepiece configured to maintain the eye of the patient in a fixed location with respect to the arcuate guide track; and a transducer connected to the carriage. The method includes emitting, from the transducer, ultrasound pulses as the carriage moves along the arcuate guide track; storing the received ultrasound pulses on a non-transitory computer readable medium; forming, by at least one electronic device, a B-Scan of the eye of the patient based on the received ultrasound pulses; binarizing, by the at least one electronic device, the B-Scan from a grayscale color palette to a black/white color palette; determining, by the at least one electronic device, an average surface of a sclera of the eye; and locating, by the at least one electronic device, a bump of the average surface of the sclera that corresponds to the scleral spur.

In U.S. patent application Ser. No. 16/422,182, a system is disclosed for detecting a scleral spur in an ultrasound image of an eye of a patient, comprising an ultrasound device, having a scan head having an arcuate guide track and a carriage movable along the arcuate guide track; an eyepiece configured to maintain the eye of the patient in a fixed location with respect to the arcuate guide track; a transducer connected to the carriage, wherein ultrasound pulses are emitted into the eye of the patient and the received ultrasound pulses are stored on a non-transitory computer readable medium; wherein at least one electronic device has non-transitory readable medium and has instructions that, when executed, cause the at least one electronic device to form a B-Scan of the eye of the patient based on the received ultrasound pulses; binarize the B-Scan from a grayscale color palette to a black/white color palette; determine an average surface of a sclera of the eye; and locate a bump of the average surface of the sclera that corresponds to the scleral spur.

The method of U.S. patent application Ser. No. 16/422,182 also discloses a system for binarizing a B-Scan of an eye of a patient, comprising an ultrasound device, having a scan head having an arcuate guide track and a carriage movable along the arcuate guide track; an eyepiece configured to maintain the eye of the patient in a fixed location with respect to the arcuate guide track; a transducer connected to the carriage, wherein ultrasound pulses are emitted into the eye of the patient, and wherein the received ultrasound pulses are stored on a non-transitory computer readable medium and wherein at least one electronic device having the non-transitory readable medium and having instructions that, when executed, cause the at least one electronic device to form a B-Scan of the eye of the patient based on the received ultrasound pulses; to determine an average intensity of a grayscale color palette of the B-Scan of the eye; to binarize the B-Scan of the eye from the grayscale color palette to a black/white color palette, wherein discrete areas of the B-Scan above a predetermined intensity are binarized to white and discrete areas of the B-Scan below the predetermined intensity are binarized to black, and the predetermined intensity depends on the average intensity.

U.S. patent application Ser. No. 16/422,182 discloses a method, using ultrasonic imaging of the anterior segment of an eye, for automatically locating the scleral spur in an ultrasound image of an eye using a form of segmentation analysis, and, using the scleral spur as a fiduciary, automatically making measurements in front of and behind the iris of an ultrasound image of an eye.

The method of U.S. patent application Ser. No. 16/422,182 is based on detecting a scleral spur in an ultrasound image of an eye of a patient. The method comprises providing an ultrasound device having a scan head with an arcuate guide track and a carriage movable along the arcuate guide track; an eyepiece configured to maintain the eye of the patient in a fixed location with respect to the arcuate guide track; and a transducer connected to the carriage. The method includes emitting, from the transducer, ultrasound pulses as the carriage moves along the arcuate guide track; storing the received ultrasound pulses on a non-transitory computer readable medium; forming, by at least one electronic device, a B-Scan of the eye of the patient based on the received ultrasound pulses; binarizing, by the at least one electronic device, the B-Scan from a grayscale color palette to a black/white color palette; determining, by the at least one electronic device, an average surface of a sclera of the eye; and locating, by the at least one electronic device, a bump of the average surface of the sclera that corresponds to the scleral spur.

Various figures and embodiments will now be discussed in connection with the processor executable functions described above under Roadmap for ICL Vault.

The whole anterior segment image is first binarized—from 0 to 255 grades of grayscale to black and white.

A. Determine background pixels and filter them out. FIG. 5 is an ultrasound image of an ICL implanted within an eye. The background pixels have been filtered out.
B. Find the approximate center by starting in a middle of image and trying to detect an iris on each side. If found, the processor executable function selects a midpoint between them, otherwise it just uses the midpoint of image.
C. For each half (horizontally flip the right side image for processing):

a. Find a lens and ICL surface point at the mid column (right side of the half image)

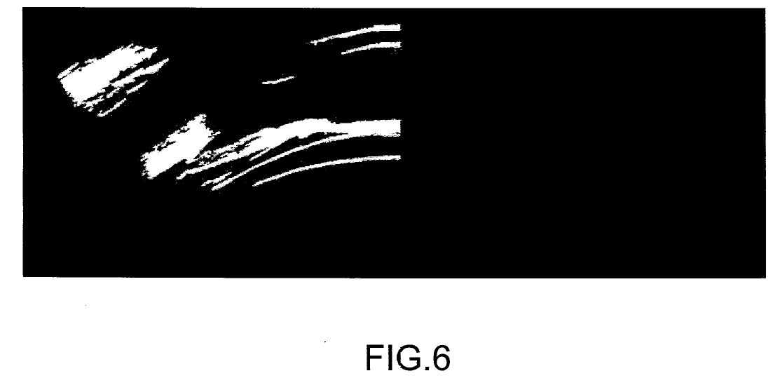

FIG. 6 is an ultrasound image of the left half of the eye from which the lens and ICL surfaces at the midpoint can be measured.

a. Trace along the surfaces in the left direction until one of the surfaces “disappears” (is no longer visible/detectable in the image.)

b. Create an object (ROI—region of interest) comprising the cavity between the 2 surfaces, bounded on the right by the edge of the half image, and on the left by the end point of the first surface to “disappear”.

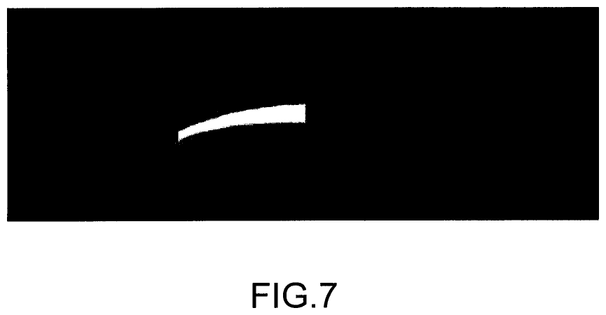

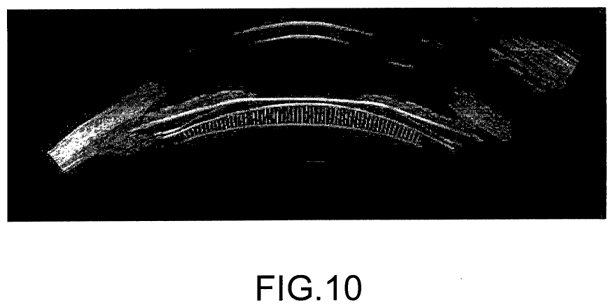

FIG. 7 is an ultrasound image showing the left-hand cavity between the 2 surfaces of FIG. 10.

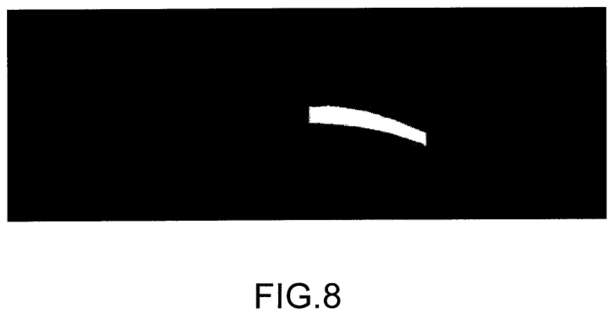

FIG. 8 is an ultrasound image showing the right-hand cavity between the 2 surfaces of an ultrasound image of the right half of the eye from which the lens and ICL surfaces at the midpoint can be measured.

D. Combine the 2 ROIs into one

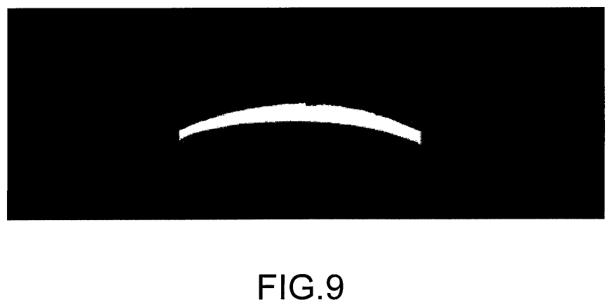

FIG. 9 is an ultrasound image of the left and right hand surfaces of FIGS. 7 and 8 (the 2 ROIs) are combined.

E. For each column in the ROI of FIG. 9, choose the bottom point of the ROI, find the point along a line perpendicular to the bottom point that intersects the top surface of the ROI. Record in memory the bottom point and top point for each column, along with the distance between the 2.

FIG. 10 is an ultrasound image showing the bottom point and top point for each column, along with the distance between the two.

1) Features of an ideal image:

a. a strong difference in pixel intensity between background pixels and real pixels sometimes in oversaturated images, surfaces blur together;

b. continuous lines defining lens and ICL surface. (Currently, as soon as a break in the surface is encountered, it is assumed that it is the end point. Or worse, a different surface is detected and interpreted as the continuation of the surface;

c. image mostly centered. Processor executable code is available detect the center if a clear definition of the iris is obtained, but this only works best when the image is mostly centered; and

d. minimal extraneous “junk” can be confused with lens or ICL, or throw off calculations of background pixel intensity.

2) Features of images causing difficulty so far:

a. Broken surfaces

Scan set 3.4 showing broken ICL/lens, resulting in mistaking the cornea for the lens.

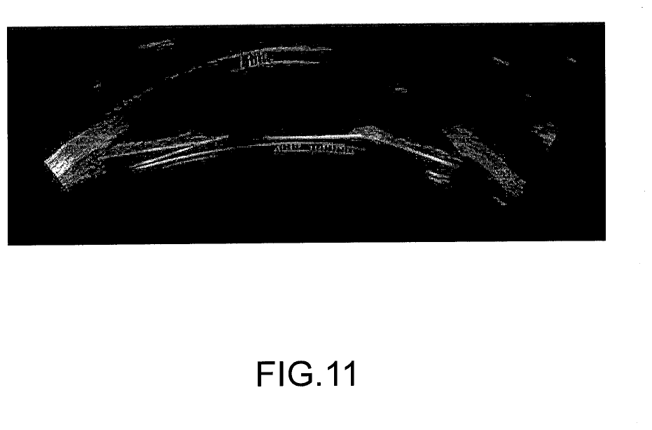

FIG. 11 is an ultrasound image illustrating features of images causing difficulty by showing broken ICL/lens that can be interpreted as the cornea mistaken for a lens.

Scan sets 2.0-2.1 showing missing ICL surface resulting in mistaking the top of the ICL for the bottom of the ICL.

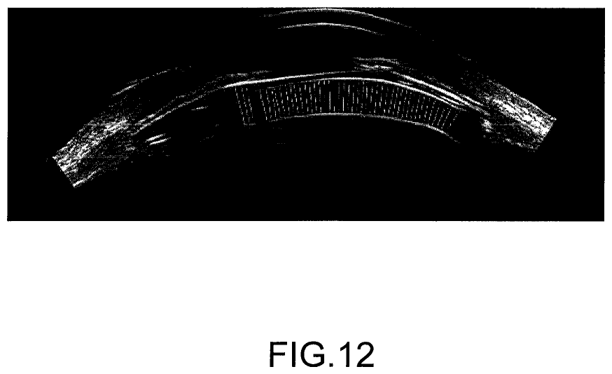

FIG. 12 is an ultrasound image illustrating features of images causing difficulty by showing a top of an ICL that was mistaken as the bottom of the ICL.

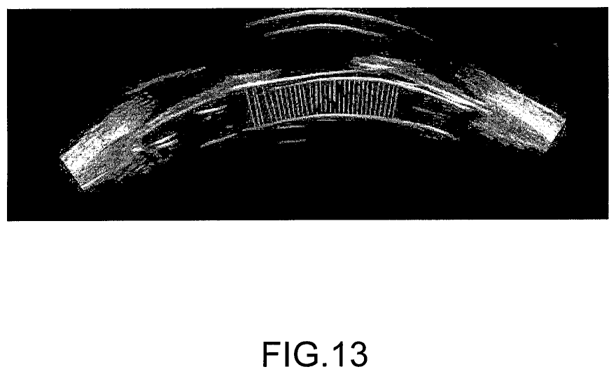

FIG. 13 is another ultrasound image illustrating features of images causing difficulty by showing a top of an ICL that was mistaken as the bottom of the ICL.

b. Extraneous features in an image

Scan set 2.2 with extraneous features under lens, causing one to think the extraneous features were a lens or an ICL:

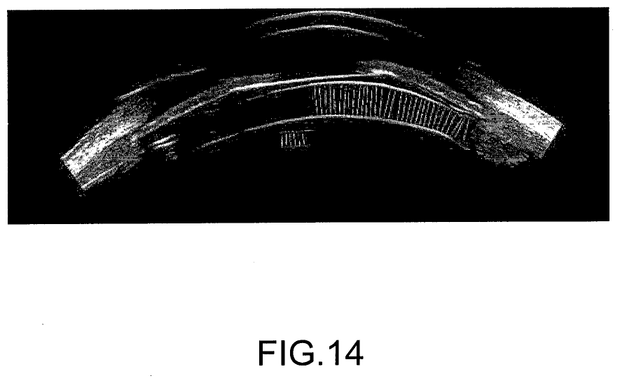

FIG. 14 is another ultrasound image illustrating features of images causing difficulty by showing a top of an ICL that was mistaken as the bottom of the ICL.

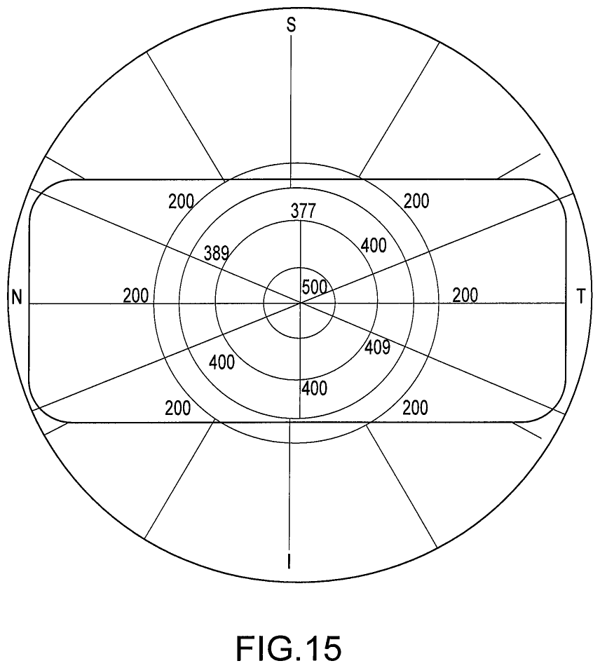

FIG. 15 is a vault map for an ICL implanted in an eye. In this figure, “N” stands for Nasal, “T” stands for Temporal, “S” stands for Superior and “I” stands for Inferior. The grey rectangle represents the ICL looking down the central axis of the eye. The numbers in the grey rectangle are measurements in microns of the distance between the anterior lens capsule and the posterior ICL along lines drawn perpendicular to the local surface of the lens capsule where the measurements are made along parallel lines at various distances from the central axis. Alternately, the numbers in the grey rectangle can be measurements in microns of the distance between the anterior lens capsule and the posterior ICL along lines drawn parallel to the central axis of the eye.

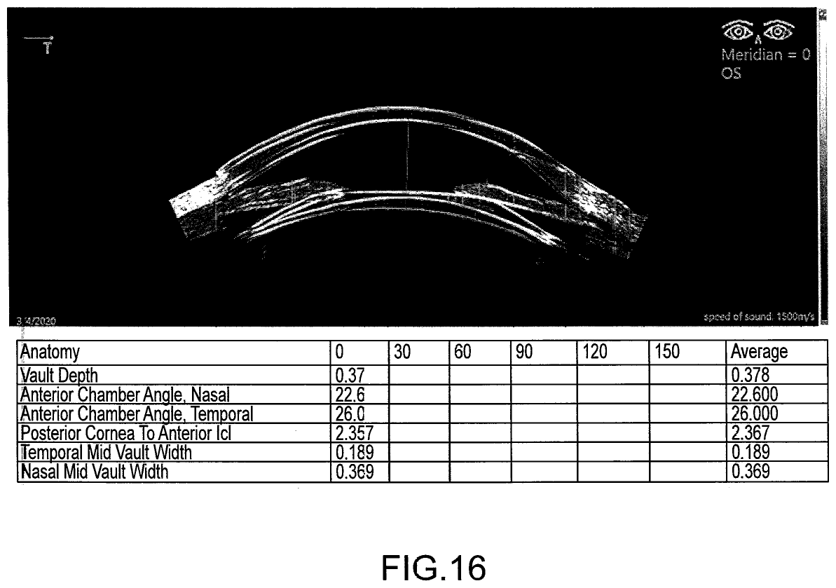

FIG. 16 is a post-op dry run. This is a B-scan taken after an ICL has been implanted in an eye. Measurements taken are shown under the B-scan and include a vault depth of 0.378 mm.

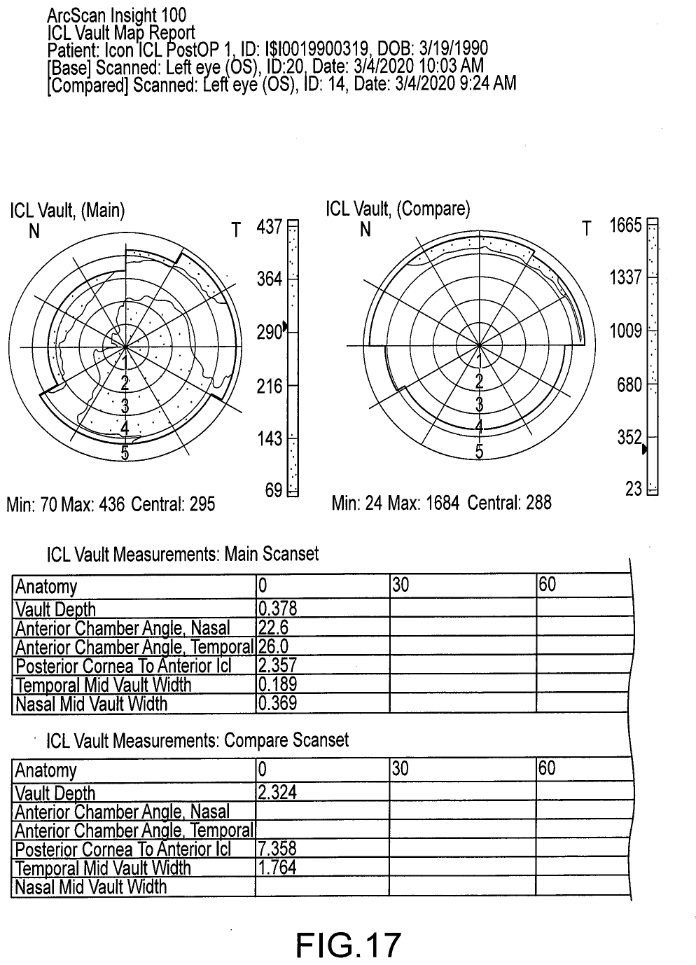

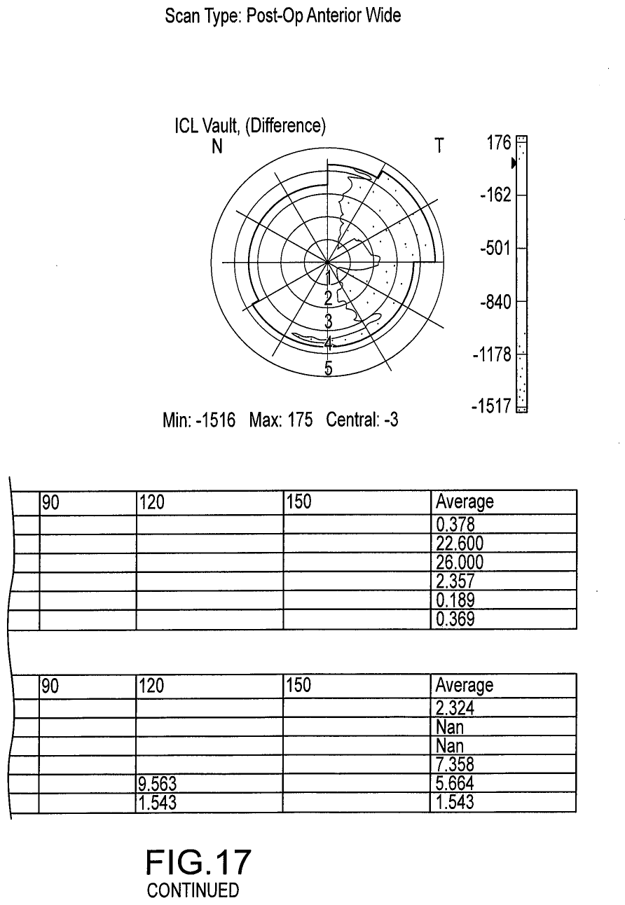

FIG. 17 is an ICL vault map report that is part of the ArcScan Insight 100 report developed from the B-scan of FIG. 16.

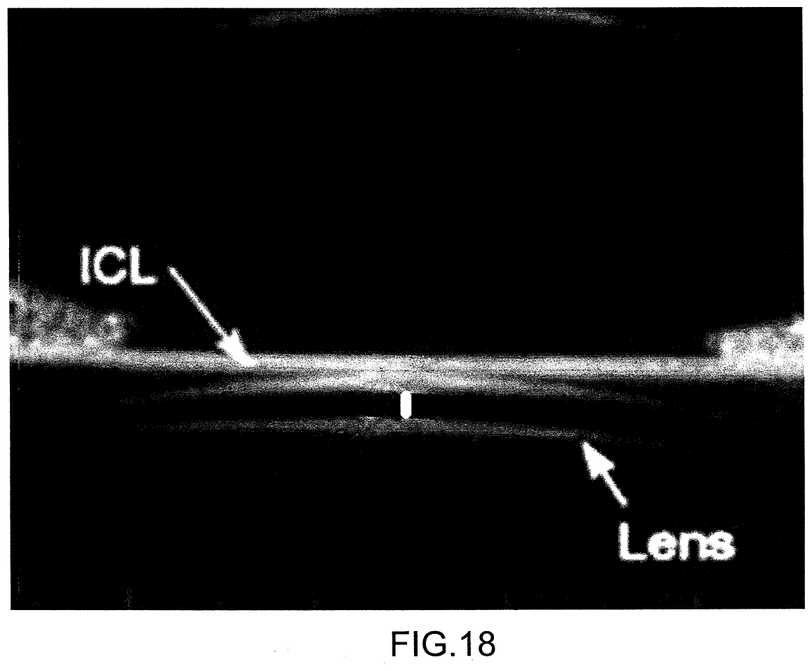

FIG. 18 is an ultrasound image of an ICL implanted in an eye. This figure shows the ICL positioned between the iris and the front of the natural lens. The vault, which is the distance between the posterior surface of the ICL and the anterior surface of the natural lens, is shown as a short white vertical line.

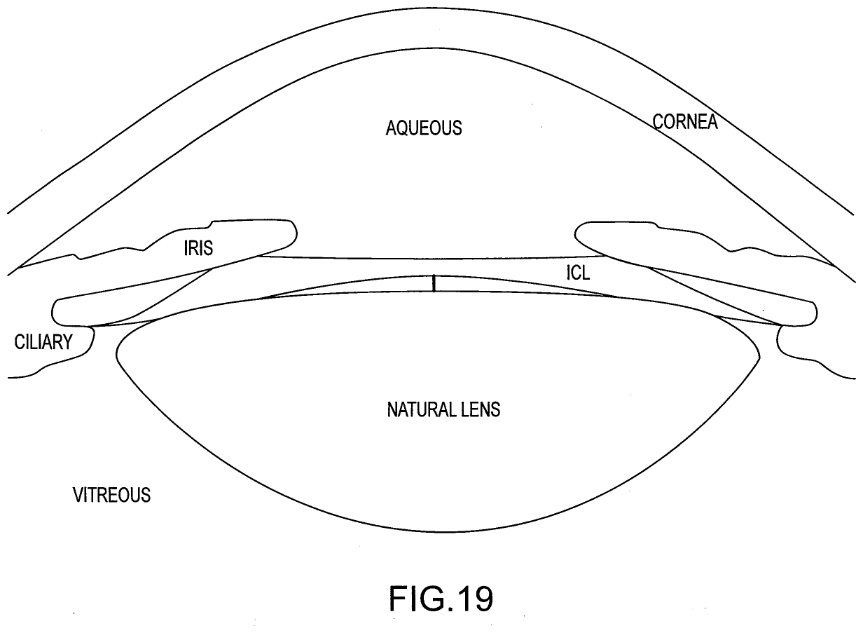

FIG. 19 is a schematic representation of an ICL as would be positioned in an eye. This figure illustrates an ICL positioned between the iris and the front of the natural lens. The vault is shown as a short black vertical line.

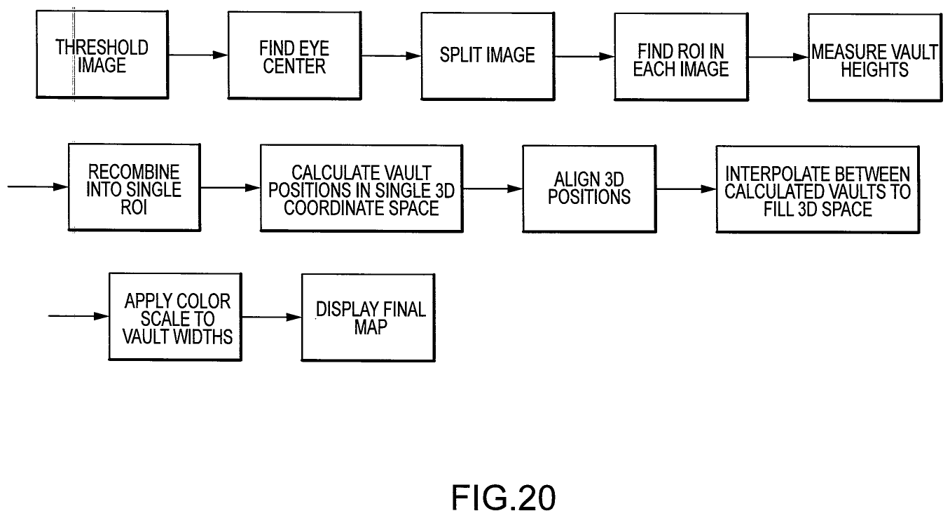

FIG. 20 is a flow chart of the principal steps used to generate a vault map from an ultrasound B-scan.

With reference to FIG. 20, the steps are as follows once an image of the eye along a certain meridian has been acquired:
1. Threshold the image to remove background pixels;
2. Find the center of the iris;
3. Split the image along the iris center (mirror the right half of the image so that it can process the same way as the left);
4. Find the region of interest (ROI) which represents a rough estimate of the vault;
5. Measure the vault height along the anterior capsule;
6. Recombine the halves to form a single Vault Map ROI for the meridian;
7. Extrapolate the positions of the ROIs for each meridian onto a single 3D coordinate system;
8. Align the ROIs within the 3D coordinate system;
9. Interpolate between calculated ROI data to fill in the 3D space between scanned meridians;
10. Assign color values to the range of virtual heights; and
11. Generate the vault map.

### 1. Thresholding.

Starting with an ultrasound image of an eye containing an ICL at a certain meridian (see FIG. 5) a threshold is determined by the processor which optimally separates the background of the image from the primary anatomy of the eye and ICL for further analysis. Those familiar with the art will recognize that there are a multitude of algorithms that can be used for finding the optimal threshold. The thresholding results in a binary image with the relevant anatomy identified.

2. Find the center of the iris.

The center of the iris is found as described in U.S. patent application Ser. No. 16/422,182, which is incorporated herein by this reference.

To find the approximate center of the iris, the processor starts with the center of the binarized image, and look at the pixels in that column of the image. Then the processor moves right or left until an area that is between the “left and right iris” depicted in the image can be identified. This is done by the processor looking for “thin” lines along the column in the binarized image, depicting the ICL and lens surfaces, without finding the “thicker” lines that would be found depicting the iris. Then the processor moves left and right until the thicker lines near the ICL surface are found, depicting the iris on each side. Once the inner edges of the iris are detected, the processor chooses the point mid-way between the two as the approximate center of the iris.

3. Split image

Once the center of the iris is found the image is split in a left and right side and the right image is horizontally flipped by the processor to allow for the same ROI algorithm to be applied (FIG. 10). This step is included for efficiency, but is not necessary as will be apparent to those skilled in the art.

### 4. Find ROI

In this step, the processor through executing the algorithm aims to identify the anterior capsule surface as well as the posterior ICL surface and reject all other anatomy present following thresholding such as the cornea surfaces, scleral wall, iris, as well as eye lid (if present) and cataract reflections (if present). Factors included in the decisions to include or reject objects in the image are size, shape, and location.

Following rejection of extraneous objects, the vault between the anterior capsule and the posterior lens is identified by the processor starting at the center and continued until one of the two surfaces are no longer visible. The ROI is returned for each half of the image (FIGS. 7 and 8).

5. Measure vault height

The vaults in both halves of the images are merged by the processor (FIG. 9) and used as the baseline information for the vault height calculations. The bottom and top of the ROI are used by the processor to find the nearby peaks in the ultrasound image which represent the anterior capsule surface (bottom of the vault) and the posterior ICL surface (top of the vault). It will be clear to those familiar with the art that numerous methods can be used by the processor to calculate the vault height.

For example, the vault height for a given point on the anterior capsule can be found by the processor by:
Finding the orientation of the anterior capsule surface at the given point;
Calculating the normal to this surface orientation;
Finding the intersection of the normal with the posterior surface of the ICL; and
Calculating the distance between the point on the anterior capsule and its corresponding intersection point on the posterior.

Finding the orientation of the anterior capsule in potentially noisy data may require the use of curve fitting or local linear fitting on capsular peak points found.

This process is repeated by the processor for all points found on the anterior capsule.

### Post Processing

In addition to the basic vault height calculations described above, additional post-processing is necessary to address issues with images that are not ideal.

Features of ideal image:

a. Strong difference in pixel intensity between background pixels and anatomical pixels.

b. Continuous lines defining lens and ICL surface.

c. Image mostly centered.

d. Minimal extraneous artefacts, such as eyelids, that may throw off thresholding.

Examples of features of images requiring additional processing:

a. Interrupted surfaces

When either the anterior capsule surface or the posterior ICL surface is interrupted, the processor addresses the gap in many ways. Small gaps can be interpolated. Gaps in the posterior ICL surface can cause the algorithm to initially jump to the superior ICL surface for the vault calculation. The processor can detect and correct these issues.

b. “Extraneous” features in an image

Small features such as those due to early stages of cataract in the images can exceed the threshold and interfere with the algorithm for detecting the anterior capsule. The processor can detect these artefacts for reliable vault detection.

FIG. 14 is another ultrasound image illustrating features of images requiring attention by the algorithm because some cataract features inside the natural lens were mistaken as part of the vault.

### Vault Maps

Once vault heights are determined for each of the images of a multi-meridian ultrasound scan, the vault height measurements can be combined into a vault map. A vault map represents the vault height as a function of the location on the anterior capsule presented in the form of a map.

FIG. 15 is a schematic vault map for an ICL implanted in an eye. In this figure, “N” stands for Nasal, “T” stands for Temporal, “S” stands for Superior and “I” stands for Inferior. The grey rectangle represents the ICL looking down the central axis of the eye. The numbers in the grey rectangle are measurements in microns of the distance between the anterior capsule and the posterior ICL along lines drawn perpendicular to the local surface of the anterior capsule where the measurements are made along parallel lines at various distances from the central axis. Alternately, the numbers in the grey rectangle can be measurements in microns of the distance between the anterior lens and the posterior ICL along lines drawn parallel to the central axis of the eye.

Those skilled in the art can appreciate there are multiple ways to create maps from a set of meridians. In this disclosure, the process detailed below is followed:

1. Recombine, by the processor, the halves to form a single Vault Map ROI for the meridian.
This reverses the initial split we performed on the image. The result is a single ROI for each meridian. This step is not necessary if the ROI wasn't split initially.
2. Extrapolate, by the processor, the positions of the ROIs for each individual B-Scan onto a single 3D coordinate system. Convert, by the processor, the position of the ROI from its two-dimensional position within the image into a common three-dimensional space based on the orientation of the scan head relative to the eye as the sweep was captured. The 3D position of each point in each ROI, for each meridian, is calculated.
3. Align, by the processor, the ROIs within the 3D coordinate system around a central axis. It is necessary to account for eye movement which will naturally occur as scanning is performed. The central axis of each ROI is calculated and used to position it within the 3D coordinate space.
4. Interpolate, by the processor, between calculated ROI data to fill in the 3D space between scanned meridians.

The full three-dimensional area of the vault must now be interpolated between the 2D data we have aligned within the common 3D space. This is done by creating, by the processor, a blank map image and aligning, by the processor, the center of the map to the central axis of the aligned ROIs. Then determine, by the processor, the radial position of each pixel relative to center and calculate, by the processor, the vault height at each pixel location. See FIG. 19 for a visual of this overlay.

Each pixel in the map will have an ROI on either side of it. If data is not present in one or both of the aligned ROIs at that pixel's radial position, the pixel will not have a height assigned to it. Interpolation is first performed within the meridians on either side of the pixel to calculate the height within the ROI at the pixels radial distance. The weighted average of the resulting values, based on the arc length between the pixel and the meridians on either side, is calculated to find the eight at the pixel's location. This process is repeated by the processor for all pixel locations we will display on our final map.

5. Assign, by the processor, color values to the height range.

Once the heights are calculated, the maximum and minimum width values are used, by the processor, to set the color scale, unless the user has created default maximum and minimum height values which will be used instead. There are a variety of options within the software to change the color scale.

### 6. Display the map.

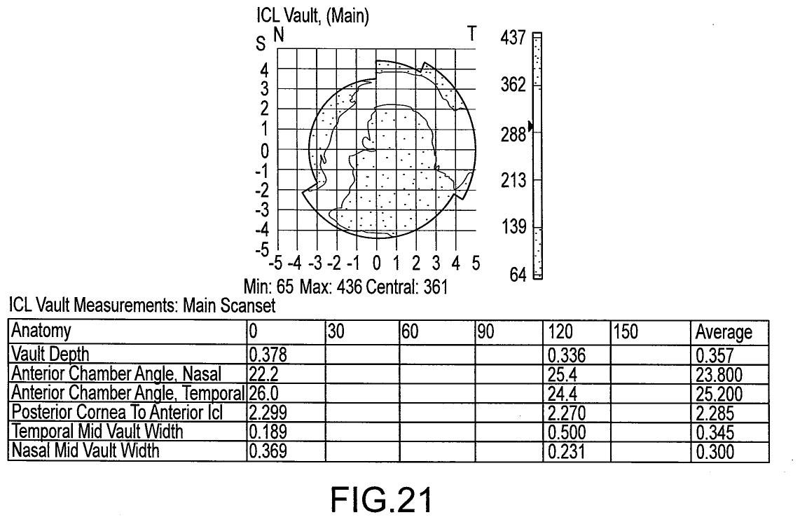

FIG. 21 is an example of a final vault map.

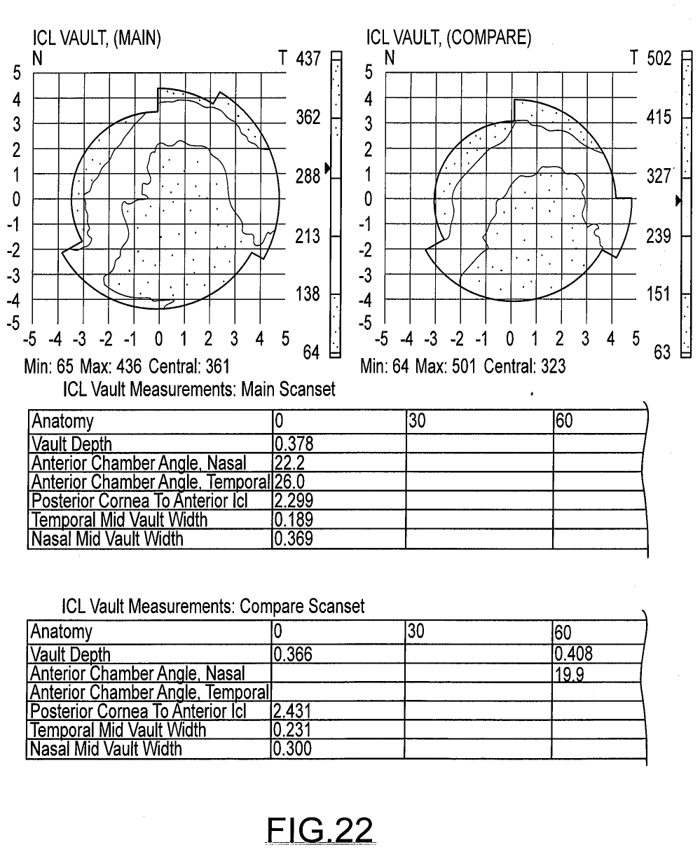

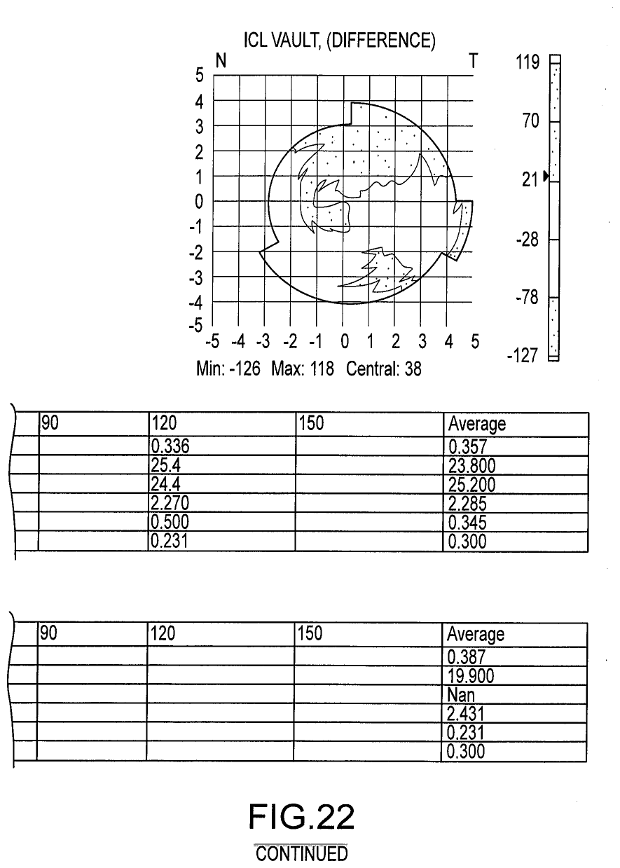

FIG. 22 shows other examples of final vault maps.

### Automated Centering and Range Finding

Early practice in preparing to set up a particular B-scan, centering the transducer on the arcuate guide track and on the center of the patient's eye was a manual process using both optical and ultrasound techniques. As disclosed in U.S. Pat. No. 8,758,252 “Innovative Components for an Ultrasonic Arc Scanning Apparatus”, FIG. 3 shows an optical video camera 323 which may be used by the operator of the arc scanner to monitor the position of the patient's eye and to determine whether the patient's eye is open before a scan is initiated.

In early practice, range finding (setting the focal point of the ultrasound transducer at the desired location within the eye) was also a manual process.

In current practice, centering and range finding has been automated. The general steps for automating centering and range finding are:

For each meridian:
1. Use, by the processor, the camera to do coarse centering
2. Do, by the processor, range finding
3. Use, by the processor, linear scans to do fine centering
   These 3 steps take about 7 seconds.

### Optical Centering

The processor takes 5 frames (about 1 second or 200 ms per frame) finds OD of iris by looking for pixel gradient, fits a circle to the horizontal diameter (to avoid eyelid blocking) by least squares, averages the diameters from the 5 frames and get average x-y of center or get x-y of center of each, and looks for movement by taking the difference of the x-y of the centers.

The processor moves the transducer to center by using the positioner and assumes the transducer is centered on the arcuate and linear tracks.

Note—on the screen, the cross hairs indicate the position of the transducer when centered. The idea is to get the center of the eye as measured to be co-incident with the cross hairs just before scanning. If the center of the cross hairs is within the diamond on the screen, then there is enough travel on the gamma and arcuate guide tracks to avoid hitting the container walls.

### Range Finding

The positioner, in response to commands from the processor, sets the scan head as far back as it can. Then the device does a horizontal linear scan (transducer at the center of the arcuate guide track). The processor looks for curved surface with Rc in the range of cornea (another least squares fit) and hence finds a z-position of cornea. The processor advanced z by say, 4 mm and repeat until anterior surface of cornea is confirmed and sets a position of focal plane where desired for scanning. If centering, the processor sets a focal plane in the cornea (about 2.5 mm beyond anterior surface).

### Ultrasound Centering

The processor sets the transducer focal plane in cornea and does a linear scan to find max of anterior cornea (shortest time from A-scan). This is the center of cornea and used to move the positioning mechanism assembly until center of cornea is co-incident with center of cross hairs.

### Automated Detection of Anterior Capsule for the Purpose of Automatically Setting Scan Depth.

For optimal image quality in ultrasound it is important to have the focal plane of the ultrasound transducer set at or near the structures of interest. One common structure of interest is the anterior capsule. For instance, measurements to correctly size an ICL are generally made between structures very close to the anterior capsule. It can be challenging to determine the optimal focal depth to image the anterior capsule when setting imaging parameters manually. Therefore, a new method was developed to automatically detect the anterior capsule using progressive scanning and ultrasound image analysis followed by the determination of the optimal focal plane depth.

The process starts with a lateral (also known as a linear) sweep with the transducer retracted to its base plane (Z=0 mm). An image analysis is performed which searches for circular surfaces in the image within a specified radius range. An analysis is performed, by the processor, to select the most likely candidate representing the epithelial surface of the cornea. If no qualifying surface is found, the transducer is moved forward a short distance (e.g., Z=2 mm) and the process is repeated with the new focal plane. Once an image is obtained with an identifiable epithelial surface, the image is further examined, by the processor, in the region posterior to the epithelial surface. A point is chosen, by the processor, at 3 mm posterior to the center of the epithelial surface, and the point among candidate surfaces with the minimal distance from the reference point is chosen, by the processor, as the target focal position. If no candidate surface within 1 mm is located, the transducer is moved forward and the process is repeated. If a capsule surface is found by the processor, the transducer is moved to that position, the lateral scan is repeated, and the same capsule analysis is performed, by the processor, again at the new focal plane, in order to adjust the focal position based on the better focused image. The transducer can now be moved to a depth (Z) such that the planned focal plan will go through the anterior capsule surface at the estimated visual axis (or a settable offset relative to that).

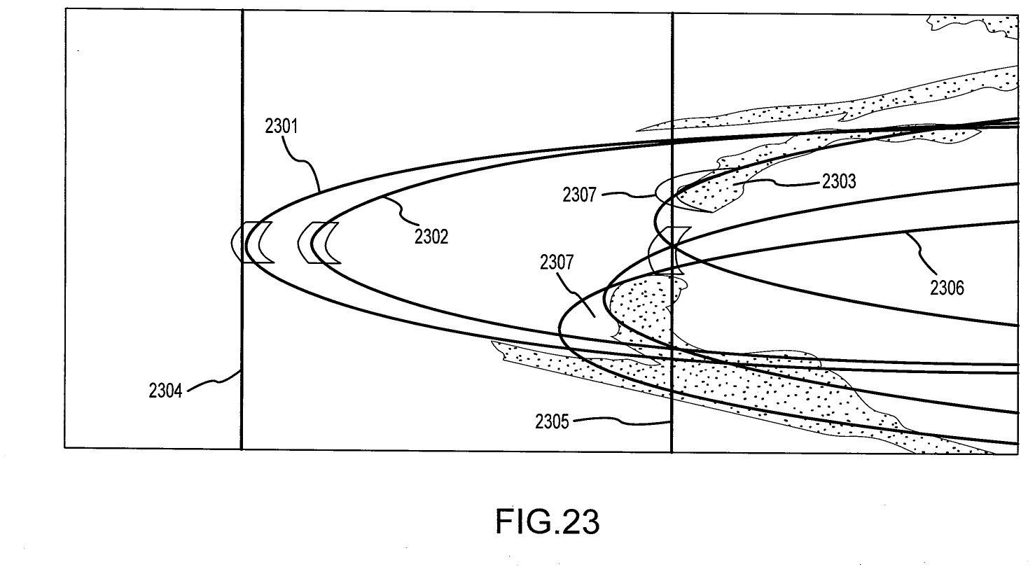

FIG. 23 is an ultrasonic image using a lateral sweep with circular surfaces detected and analyzed, by the processor, indicating the epithelial and endothelial surfaces of the cornea (2301), a secondary candidate surface (2302), a number of circular surfaces rejected by the cornea analysis (2306), and the final selected Z-location of the capsule on the estimated visual axis. The transducer can now be moved, by the processor, such that the planned focal plan will go through the anterior capsule surface at the estimated visual axis (or a settable offset relative to that). Feature 2307 indicates the nearest detected surface to the transducer, which defines a safety boundary to which the transducer should not be moved. Feature 2304 is a line representing the starting Z-coordinate for the detection algorithm. Feature 2305 is a line representing the desired Z-coordinate for the transducer focal point. The posterior iris 2303 is also shown.

In one aspect of the present disclosure, a method for detecting and measuring a vault of an anterior segment of an eye of a patient can comprise: imaging, by an ultrasound scanning device, an anterior segment of the eye; locating, by a processor, an implanted contact lens (ICL) between a cornea and a natural lens; forming, by the processor, a B-scan of at least a portion of the eye based on the received ultrasound pulses; binarizing and thresholding by the processor, the B-Scan from a grayscale color palette to a black/white color palette; determining, by the processor, using segmentation analysis of the binarized and thresholded B-scan, a fiduciary location in the anterior segment of the eye; and forming, by the processor and using the binarized and thresholded B-scan and fiduciary location, a vault map mapping a distance between an anterior segment surface and a posterior surface of the ICL along a plurality of lines drawn perpendicular to a local surface of the anterior segment surface.

In the aspect, the determining can include locating, by the processor, a center of an iris of the eye; dividing the B-Scan along the iris center to form left and right portions of the B-Scan; and horizontally flipping one of the left and right portions.

The aspect can further include for each of the left and right portions: finding, by the processor, a region of interest representing a rough estimate of a vault; measuring, by the processor, a vault height along the anterior segment surface; and merging, by the processor, the measured vault heights in the left and right portions to form a common vault map region of interest.

In the aspect, the B-scan can comprise multiple B-scans and each of the B-scans corresponds to a region of interest and wherein, for each of the plurality of regions of interest, the forming can comprise: extrapolating, by the processor, a position of each selected region of interest for each scanned meridian onto a common three-dimensional coordinate system; and aligning, by the processor, a central axis of each of the regions of interest in the plurality of regions of interest within the common three-dimensional coordinate system.

In the aspect, the forming can comprise interpolating, by the processor, between calculated region of interest data for each region of interest to fill in a three-dimensional space between scanned meridians.

In the aspect, the forming can comprise: assigning, by the processor, color values to a range of virtual heights; and generating, by the processor, the vault map based on the assigned color values.

In an aspect, an eye imaging system can comprise: an input to receive a plurality of A-scans of an anterior capsule of an eye of a patient from an ultrasound scanning device; a processor coupled with the input; and a memory coupled with and readable by the processor and storing therein a set of instructions which, when executed by the processor causes the processor to: locate an implanted contact lens (ICL) between a cornea and a natural lens of the eye; form, from the plurality of A-scans, a B-scan of the anterior capsule of the eye; binarize and threshold the B-Scan from a grayscale color palette to a black/white color palette; determine, using segmentation analysis, a fiduciary location in the anterior capsule of the eye; and form, using the binarized and thresholded B-scan and fiduciary location, a vault map mapping a distance between an anterior capsule and a posterior surface of the ICL along a plurality of lines drawn perpendicular to a local surface of the anterior capsule surface.

In the aspect, the determining can comprise: locating a center of an iris of the eye; dividing the B-Scan along the iris center to form left and right portions of the B-Scan; and horizontally flipping one of the left and right portions.

In the aspect, for each of the left and right portions, the instructions can cause the processor to: find a region of interest representing a rough estimate of a vault; and measure a vault height along the anterior capsule surface; wherein the instructions can further cause the processor to merge the measured vault heights in the left and right portions to form a common vault map region of interest.

In the aspect, the B-scan can comprise multiple B-scans and each of the B-scans can correspond to a region of interest and wherein, for each of the plurality of regions of interest, the forming can comprise: extrapolating a position of each selected region of interest for each scanned meridian onto a common three-dimensional coordinate system; and aligning a central axis of each of the regions of interest in the plurality of regions of interest within the common three-dimensional coordinate system.

In the aspect, the forming can comprise: interpolating between calculated region of interest data for each region of interest to fill in a three-dimensional space between scanned meridians.

In the aspect, the forming can comprise assigning color values to a range of virtual heights; and generating the vault map based on the assigned color values.

In an aspect of the disclosure, an eye imaging system can comprise: an input to receive a plurality of A-scans of an anterior capsule of an eye of a patient from an ultrasound scanning device; a processor coupled with the input; and a memory coupled with and readable by the processor and storing therein a set of instructions which, when executed by the processor causes the processor to: locate an implanted contact lens (ICL) between a cornea and a natural lens of the eye; form, from the plurality of A-scans, a B-scan of the anterior capsule of the eye; remove background pixels of the B-scan to form a binary image; determine a fiduciary location in the anterior capsule of the eye; and determine, from the binary image and the fiduciary location, a vault distance between the anterior capsule and a posterior surface of the ICL along a selected line drawn perpendicular to a local surface of the anterior capsule surface.

In an aspect of the disclosure, an eye imaging method can comprise: a processor, locating an implanted contact lens (ICL) between a cornea and a natural lens of the eye; forming, from the plurality of A-scans, a B-scan of the anterior capsule of the eye; removing background pixels of the B-scan to form a binary image; determining a fiduciary location in the anterior capsule of the eye; and determining, from the binary image and the fiduciary location, a vault distance between the anterior capsule and a posterior surface of the ICL along a selected line drawn perpendicular to a local surface of the anterior capsule surface.

In the aspects, the vault distance determining can comprise: forming, based on a plurality of vault distances, a vault map mapping a distance between the anterior capsule and the posterior surface of the ICL along a plurality of lines drawn perpendicular to a local surface of the anterior capsule surface, the plurality of lines comprising the selected lines.

In the aspects, the determining can comprise: locating a center of an iris of the eye; dividing the B-Scan along the iris center to form left and right portions of the B-Scan; and horizontally flipping one of the left and right portions.

In the aspects, for each of the left and right portions, the instructions can cause the processor to: find a region of interest representing a rough estimate of a vault; and measure a vault height along the anterior capsule surface; and wherein the instructions can further cause the processor to merge the measured vault heights in the left and right portions to form a common vault map region of interest.

In the aspects, the B-scan can comprise multiple B-scans and each of the B-scans can correspond to a region of interest and wherein, for each of the plurality of regions of interest, the forming can comprise: extrapolating a position of each selected region of interest for each scanned meridian onto a common three-dimensional coordinate system; and aligning a central axis of each of the regions of interest in the plurality of regions of interest within the common three-dimensional coordinate system.

In the aspects, the forming can comprise interpolating between calculated region of interest data for each region of interest to fill in a three-dimensional space between scanned meridians.

In the aspects, the forming can comprise: assigning color values to a range of virtual heights; and generating the vault map based on the assigned color values.

In the aspects, the instructions can cause the processor to: identify, based on a size, shape, and/or location of anatomical structures in the binary image, the anterior capsule surface and the posterior ICL surface; remove anatomical structures other than the anterior capsule surface and posterior ICL surface from the resulting binary image to form a region of interest; based on the region of interest, identify peaks in the resulting binary image, the peaks representing the anterior capsule surface and the posterior ICL surface; and measure a vault height along the anterior capsule surface. In the foregoing description, for the purposes of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other types of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Specific details were given in the description to provide a thorough understanding of the embodiments. However, it will be understood by one of ordinary skill in the art that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Also, it is noted that the embodiments were described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine readable medium such as storage medium. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The present disclosure, in various embodiments, includes components, methods, processes, systems and/or apparatus substantially as depicted and described herein, including various embodiments, sub-combinations, and subsets thereof. Those of skill in the art will understand how to make and use the present disclosure after understanding the present disclosure. The present disclosure, in various embodiments, includes providing devices and processes in the absence of items not depicted and/or described herein or in various embodiments hereof, including in the absence of such items as may have been used in previous devices or processes, for example for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover though the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for detecting and measuring a vault of an anterior segment of an eye of a patient comprising:
imaging, by an ultrasound scanning device, an anterior segment of the eye;
locating, by a processor, an implanted contact lens (ICL) between a cornea and a natural lens;
forming, by the processor, one or more B-scans of at least a portion of the eye based on the received ultrasound pulses;
converting, by the processor, the one or more B-scans from a grayscale color palette to a black/white color palette;
determining, by the processor, using segmentation analysis of the converted one or more B-scans, a fiduciary location in the anterior segment of the eye; and
forming, by the processor and using the converted one or more B-scans and fiduciary location, a vault map mapping a distance between an anterior segment surface and a posterior surface of the ICL along a plurality of lines drawn perpendicular to a local surface of the anterior segment surface.

2. A method of claim 1, wherein the converting comprises binarizing and thresholding the one or more B-scans to remove background image information and wherein the determining comprises:
locating, by the processor, a center of an iris of the eye;
dividing the one or more B-scans along the iris center to form left and right portions of the one or more B-scans; and
horizontally flipping one of the left and right portions.

3. The method of claim 2, further comprising:
for each of the left and right portions:
finding, by the processor, a region of interest representing a rough estimate of a vault;
measuring, by the processor, a vault dimension along the anterior segment surface; and
merging, by the processor, the measured vault dimensions in the left and right portions to form a common vault map region of interest.

4. The method of claim 3, wherein the one or more B-scans comprises multiple B-scans and each of the multiple B-scans corresponds to a region of interest and wherein, for each of the plurality of regions of interest, the forming comprises:
extrapolating, by the processor, a position of each selected region of interest for each scanned meridian onto a common three-dimensional coordinate system; and
aligning, by the processor, a central axis of each of the regions of interest in the plurality of regions of interest within the common three-dimensional coordinate system.

5. The method of claim 4, wherein the forming comprises:
interpolating, by the processor, between calculated region of interest data for each region of interest to fill in a three-dimensional space between scanned meridians.

6. The method of claim 5, wherein the forming comprises:
assigning, by the processor, color values to a range of virtual heights; and
generating, by the processor, the vault map based on the assigned color values.

7. An eye imaging system, comprising:
an input to receive a plurality of A-scans of an anterior capsule of an eye of a patient from an ultrasound scanning device;
a processor coupled with the input; and
a memory coupled with and readable by the processor and storing therein a set of instructions which, when executed by the processor causes the processor to:
locate an implanted contact lens (ICL) between a cornea and a natural lens of the eye;
form, from the plurality of A-scans, one or more B-scans of the anterior capsule of the eye;
convert the one or more B-scans from a grayscale color palette to a black/white color palette;
determine a fiduciary location in the anterior capsule of the eye; and
form, using the converted one or more B-scans and fiduciary location, a vault map mapping a height between the anterior capsule and a posterior surface of the ICL along a plurality of lines drawn perpendicular to a local surface of the anterior capsule.

8. The system of claim 7, wherein the converting comprises binarizing and thresholding the one or more B-scans to remove background image information and wherein the determining is performed using segmentation analysis.

9. The system of claim 8, wherein the determining comprises:
locating a center of an iris of the eye;
dividing the one or more B-scans along the iris center to form left and right portions of the one or more B-scans; and
horizontally flipping one of the left and right portions.

10. The system of claim 9, wherein, for each of the left and right portions, the instructions cause the processor to:
find a region of interest representing a rough estimate of a vault; and
measure a vault dimension along the anterior capsule surface; and
wherein the instructions further cause the processor to merge the measured vault dimensions in the left and right portions to form a common vault map region of interest.

11. The system of claim 10, wherein the one or more B-scans comprises multiple B-scans and each of the multiple B-scans corresponds to a region of interest and wherein, for each of the plurality of regions of interest, the forming comprises:
extrapolating a position of each selected region of interest for each scanned meridian onto a common three-dimensional coordinate system; and
aligning a central axis of each of the regions of interest in the plurality of regions of interest within the common three-dimensional coordinate system.

12. The system of claim 11, wherein the forming comprises:
interpolating between calculated region of interest data for each region of interest to fill in a three-dimensional space between scanned meridians.

13. The system of claim 12, wherein the forming comprises:
assigning color values to a range of virtual heights; and
generating the vault map based on the assigned color values.

14. An eye imaging system, comprising:
an input to receive a plurality of A-scans of an anterior capsule of an eye of a patient from an ultrasound scanning device;
a processor coupled with the input; and
a memory coupled with and readable by the processor and storing therein a set of instructions which, when executed by the processor causes the processor to:
locate an implanted contact lens (ICL) between a cornea and a natural lens of the eye;
form, from the plurality of A-scans, one or more B-scans of the anterior capsule of the eye;
remove background pixels of the one or more B-scans to form a binary image;
determine a fiduciary location in the anterior capsule of the eye; and
determine, from the binary image and the fiduciary location, a vault dimension between the anterior capsule and a posterior surface of the ICL along a selected line drawn perpendicular to a local surface of the anterior capsule surface.

15. The system of claim 14, wherein the vault distance determining comprises:
forming, based on a plurality of vault distances, a vault map mapping a distance between the anterior capsule and the posterior surface of the ICL along a plurality of lines drawn perpendicular to a local surface of the anterior capsule surface, the plurality of lines comprising the selected lines.

16. The system of claim 15, wherein the determining comprises:
locating a center of an iris of the eye;
dividing the one or more B-scans along the iris center to form left and right portions of the one or more B-scans; and
horizontally flipping one of the left and right portions.

17. The system of claim 16, wherein, for each of the left and right portions, the instructions cause the processor to:
find a region of interest representing a rough estimate of a vault; and
measure a vault dimension along the anterior capsule surface; and
wherein the instructions further cause the processor to merge the measured vault dimensions in the left and right portions to form a common vault map region of interest.

18. The system of claim 17, wherein the one or more B-scans comprises multiple B-scans and each of the multiple B-scans corresponds to a region of interest and wherein, for each of the plurality of regions of interest, the forming comprises:
extrapolating a position of each selected region of interest for each scanned meridian onto a common three-dimensional coordinate system; and
aligning a central axis of each of the regions of interest in the plurality of regions of interest within the common three-dimensional coordinate system.

19. The system of claim 18, wherein the forming comprises:
interpolating between calculated region of interest data for each region of interest to fill in a three-dimensional space between scanned meridians.

20. The system of claim 19, wherein the forming comprises:
assigning color values to a range of virtual heights; and
generating the vault map based on the assigned color values.

21. The system of claim 14, wherein the instructions cause the processor to:
identify, based on a size, shape, and/or location of anatomical structures in the binary image, the anterior capsule surface and the posterior ICL surface;
remove anatomical structures other than the anterior capsule surface and posterior ICL surface from the resulting binary image to form a region of interest;
based on the region of interest, identify peaks in the resulting binary image, the peaks representing the anterior capsule surface and the posterior ICL surface; and
measure a vault dimension along the anterior capsule surface.
